(19) United States
(12) Patent Application Publication
(10) Pub. No.: US 2009/0181052 A1
(43) Pub. Date: Jul. 16, 2009

(54) VACCINE

(75) Inventor: Brigitte Desiree Alberte Colau, Rixensart (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GlaxoSmithKline Biologicals sa

(21) Appl. No.: 11/912,687

(22) PCT Filed: Apr. 24, 2006
(86) PCT No.: PCT/EP06/03918

§ 371 Date: Jun. 12, 2008

Related U.S. Application Data
(60) Provisional application No. 60/674,829, filed on Apr. 26, 2005.

(30) Foreign Application Priority Data
May 3, 2005 (GB) 0509010.5
Jun. 14, 2005 (EP) PCT/EP2005/006461

(51) Int. Cl. A61K 39/12; A61P 31/20

(52) U.S. Cl. 424/205.1

(57) ABSTRACT

A method for prevention of HPV infection and/or disease, the method comprising delivery of a first HPV vaccine comprising an L1 protein or immunogenic fragment thereof from at least HPV 16 and HPV 18, and a second HPV vaccine which does not comprise the HPV 16 and HPV 18 L1 components from the first vaccine, and which second vaccine comprises an L1 protein or immunogenic fragment thereof from at least one other oncogenic HPV type, wherein the first and second vaccines may be delivered in either order and delivery is separated by a suitable time interval.

### BACKGROUND OF THE INVENTION

Papillomaviruses are small DNA tumour viruses, which are highly species specific. So far, over 100 individual human papillomavirus (HPV) genotypes have been described. HPVs are generally specific either for the skin (e.g. HPV-1 and -2) or mucosal surfaces (e.g. HPV-6 and -11) and usually cause benign tumours (warts) that persist for several months or years. Such benign tumours may be distressing for the individuals concerned but tend not to be life threatening, with a few exceptions.

Some HPVs are also associated with cancers, known as oncogenic HPV types. The strongest positive association between an HPV and human cancer is that which exists between HPV-16 and HPV-18 and cervical carcinoma. Cervical cancer is the most common malignancy in developing countries, with about 500,000 new cases occurring in the world each year.

Other HPV types which can cause cancer are types 31, 33, 35, 39, 45, 51, 52, 56, 58, 59, 66 and 68. Types 16 and 18 are those which have the highest association with cervical cancer. Types 31 and 45 are the types with the next highest association with a cancer risk (Munoz N, Bosch F X, de Sanjose S et al. International Agency for Research on Cancer Multicenter Cervical Cancer Study Group. N Engl J Med 2003; 348: 518-27.)

HPV virus like particles (VLPs) have been suggested as potential vaccines for treatment of HPV. Animal studies have shown that VLPs produce no cross protection against infection for other HPV types—see, for example Suzich, J. A., et al, Proc Natl Acad Sci, 92: 11553-11557, 1995, and Breitburd, Seminars in Cancer Biology, vol 9, 1999, pp 431-445.

WO2004/056389 discloses that an HPV 16, 18 VLP vaccine can provide cross protection against infection by HPV types other than 16 and 18. Statistically significant protection was observed against certain groups of HPV types.

There is still a need for a vaccine that protects against multiple HPV types.

### STATEMENT OF INVENTION

The present invention relates to a vaccination schedule for protection against HPV infection and/or disease, the schedule comprising delivery of a first HPV vaccine comprising an L1 protein or immunogenic fragment thereof from at least HPV 16 and HPV 18, and a second HPV vaccine which does not comprise the HPV 16 and HPV 18 L1 components from the first vaccine, and which second vaccine comprises an L1 protein or immunogenic fragment thereof from at least one other oncogenic HPV type, wherein the first and second vaccines may be delivered in either order and delivery is separated by a suitable interval.

The present invention further relates to a vaccination schedule for protection against HPV infection or disease, the schedule comprising delivery of a first HPV vaccine comprising an L1 protein or immunogenic fragment thereof from at least HPV 16 and HPV 18, and after a suitable interval a second HPV vaccine which does not comprise the HPV 16 and HPV 18 L1 components from the first vaccine, and which second vaccine comprises an L1 protein or immunogenic fragment thereof from at least one other oncogenic HPV type.

The invention further relates to a method for prevention of HPV infection and/or disease, the method comprising delivery of a first HPV vaccine comprising an L1 protein or immunogenic fragment thereof from at least HPV 16 and HPV 18, and a second HPV vaccine which does not comprise the HPV 16 and HPV 18 L1 components from the first vaccine, and which second vaccine comprises an L1 protein or immunogenic fragment thereof from at least one other oncogenic HPV type, wherein the first and second vaccines may be delivered in either order and delivery is separated by a suitable time interval.

The invention also relates to a method for prevention of HPV infection and/or disease, the method comprising delivery of a first HPV vaccine comprising an L1 protein or immunogenic fragment thereof from at least HPV 16 and HPV 18, and after a suitable interval a second HPV vaccine which does not comprise the HPV 16 and HPV 18 L1 components from the first vaccine, and which second vaccine comprises an L1 protein or immunogenic fragment thereof from at least one other oncogenic HPV types.

The invention also relates to vaccine compositions of the invention per se.

The invention also relates to kits comprising the first and second vaccine compositions of the invention.

In one aspect the invention relates to use of an HPV L1 protein or immunogenic fragment thereof from one first HPV type in the preparation of a medicament for boosting the immune response generated by an HPV L1 protein or immunogenic fragment thereof from a second, different HPV type. In one aspect the L1 protein or fragment thereof from the second HPV type is in the form of a virus like particle and is used to boost the response generated by the L1 protein or fragment thereof from the first HPV type also in the form of a virus like particle.

The invention also relates to the use of HPV L1, preferably in the form of VLPs, from HPV strains which are not HPV 16, in boosting the response to HPV 16.

The invention also relates to the use of L1, preferably in the form of VLPs, from HPV strains which are not HPV 18, in boosting the response to HPV 18.

In one aspect the strains for boosting the immune response are those for which some level of cross protection is observed in Example 1, such as HPV 31, HPV 45 and HPV 52.

### FIGURES

FIGS. 1 and 5 illustrate the antibodies generated to HPV 16 with different prime and boost regimes.

FIGS. 2 and 6 illustrate the antibodies generated to HPV 18 with different prime and boost regimes.

FIGS. 3 and 7 illustrate the antibodies generated to HPV 31 with different prime and boost regimes.

FIGS. 4 and 8 illustrate the antibodies generated to HPV 45 with different prime and boost regimes.

FIG. 9 illustrates a summary of the antibody responses in FIGS. 1-8.

FIGS. 10 and 14 illustrates the % HPV16 L1 specific T cells in the CD4+ population with different prime and boost regimes.

FIGS. 11 and 15 illustrates the % HPV18 L1 specific T cells in the CD4+ population with different prime and boost regimes.

FIGS. 12 and 16 illustrates the % HPV31 L1 specific T cells in the CD4+ population with different prime and boost regimes.

FIGS. 13 and 17 illustrates the % HPV45 L1 specific T cells in the CD4+ population with different prime and boost regimes.

FIG. 18 illustrates a summary of the CMI data in FIGS. 10-13.

### DETAILED DESCRIPTION

The general existence of cross protection afforded by HPV 16 and HPV 18 against both incident and persistent infection, as assessed in relation to certain groups of HPV types, has been disclosed in WO2004/056389.

We have surprisingly discovered that the cross protection against certain (non HPV16, HPV 18) HPV types (as assessed by the efficacy of an HPV 16 and HPV 18 vaccine against those types), is higher than against certain other (non HPV16, HPV 18) HPV types. Cross protection may be considered as the protection afforded by a vaccine containing one HPV type against infection (incident or persistent) and/or disease caused by a different HPV type. Cross protection may be assessed by considering the vaccine efficacy (V.E.), wherein the V.E. is the % improvement in protection against infection or disease by the vaccine compared to a placebo group for a given type.

Infection may be incident or persistent infection. Disease may be abnormal cytology, ASCUS, CIN1, CIN2, CIN3 or cervical cancer related to HPV infection. Infection may be assessed by PCR, for example. Disease may be assessed by histological examination or analysis of biomarkers such as p16.

Some level of cross protection is considered to be present when the cross protection is statisitically significantly higher than that given by a placebo. Suitably the level of cross protection is greater than 0% and up to 20%, up to 40%, up to 60%, up to 70%, up to 80%, or greater than 80%, as measured by the vaccine efficacy of an HPV vaccine against infection or disease caused by an HPV type not present in the vaccine, when compared to a placebo.

Such a finding has potential implications for vaccine design and the design of vaccination schedules.

For example, types for which cross protection is observed might be used as a prime or boost for one another, after a suitable interval, as well as providing homologous protection, and may allow the number of vaccinations for any given HPV type to be reduced.

The present invention relates to a vaccination schedule for protection against HPV infection or disease, the schedule comprising delivery of a first HPV vaccine comprising an L1 protein or immunogenic fragment thereof from at least HPV 16 and HPV 18, and a second HPV vaccine which does not comprise the HPV 16 and HPV 18 L1 components from the first vaccine, and which second vaccine comprises an L1 protein or immunogenic fragment thereof from at least one other oncogenic HPV type, wherein the first and second vaccines may be delivered in either order and delivery is separated by a suitable interval.

A suitable interval may be 7 days, 2 weeks, 4 weeks, 6 weeks, 8 weeks, 3 months, 4, months, 5 months, 6 months, 1 year. Vaccination schedules might then be 0, 1 months, or 0, 2 months, or 0, 4 months or 0, 6 months, for example. Generally a suitable interval is one in which the boosting effect of the vaccine delivered second can be observed, for example by measurement of antibody titres or cell mediated immunity. Generally a suitable interval is one in which the primary immune response has been provoked by delivery of a vaccine, as measured by serum antibody titres for example, before delivery of a second vaccine. In one aspect a suitable interval is shortly after the peak of the primary response, typically the antibody response. In one aspect this interval is from 2-26 weeks after the initial vaccination, suitably 2-22 weeks, 2-18 weeks, 2-14 weeks, 2-12 weeks, 2-10 weeks, 2-8 weeks, 2-6 weeks, and in one aspect 1 month after the initial vaccination.

Oncogenic HPV types include HPV 31, 33, 35, 39, 45, 51, 52, 56, 58, 59, 66 and 68.

Suitably cross protection is observed between at least one component of the first vaccine and one component of the second vaccine. As such, the first vaccine suitably comprises an L1 protein or immunogenic fragment thereof from HPV 16 and HPV 18 and the second vaccine suitably comprises an L1 protein or immunogenic fragment thereof from HPV 31, or HPV 45 or HPV 52.

In one aspect a first vaccine comprises L1 protein or immunogenic fragment thereof from HPV 16 and HPV 18. In a further aspect the first vaccine includes L1 protein or immunogenic fragment thereof from additional HPV types, such as one or more of HPV types 31, 33, 35, 39, 45, 51, 52, 56, 58, 59, 66, 68. In one aspect the HPV type is HPV 33.

In another aspect the second vaccine excludes at least the HPV 16 and 18 antigens from the first vaccine and comprises an L1 protein or immunogenic fragment thereof from an HPV type shown to have a cross protective interaction with HPV 16 and/or HPV 18 herein, suitably HPV 31 and/or 45 and/or 52. The second vaccine may include additional types such as one or more of HPV 31, 33, 35, 39, 45, 51, 52, 56, 58, 59, 66, 68.

In one aspect the second vaccine comprises L1 protein or immunogenic fragment thereof from HPV 31, and/or HPV 45. In a further aspect the second vaccine includes L1 protein or immunogenic fragment thereof from type 52.

In a further aspect the second vaccine includes L1 protein or immunogenic fragment thereof from type 58.

The invention thus relates to a vaccine composition comprising an L1 protein or immunogenic fragment thereof from HPV 31 but excluding an L1 protein or immunogenic fragment thereof from at least HPV 16 and HPV 18.

The invention also relates to a vaccine composition comprising an L1 protein or immunogenic fragment thereof from HPV 45 but excluding an L1 protein or immunogenic fragment thereof from at least HPV 16 and HPV 18.

The invention also relates to a vaccine composition comprising an L1 protein or immunogenic fragment thereof from HPV 52 but excluding an L1 protein or immunogenic fragment thereof from at least HPV 16 and HPV 18.

In another aspect the second vaccine excludes all the HPV L1 containing antigens from the first vaccine. Suitably the first and second vaccines share no identical L1 proteins or identical complete protein fragments. For the avoidance of doubt, there may be regions within the L1 proteins or L1 fragments used in the first and second vaccine that are similar or identical. However use of identical antigens, the antigen being either whole L1 or a fragment thereof, in both the first and second vaccine is not preferred in this aspect.

In one aspect the first and second vaccines do not share an HPV L1 protein or immunogenic fragment thereof from the same HPV type.

In one aspect the components of the first vaccine contain HPV species which are phylogenetically not closely related, such as HPV 16 and HPV 18.

In one aspect the components of the second vaccine contain HPV species which are phylogenetically not closely related, such as HPV 31 and HPV 45.

Phylogenetically not closely related types are in different species groups as assessed by de Villiers et al. Virology. 2004 June 20; 324(1):17-27.

Suitably the HPV 16 and/or HPV 18 components in the first vaccine component protect against HPV infection and/or disease caused by at least one HPV type in the second vaccine.

Suitably the first and second vaccines of the invention comprise HPV L1 VLPs from the following HPV types:
first vaccine a combination of HPV 16, HPV 18 and HPV 33. The first vaccine may contain additionally HPV 58.
second vaccine a combination of HPV 31, HPV 45 and optionally HPV 52 and/or HPV 58.

Suitably the vaccine delivered second boosts the immune response against at least one component in the vaccine delivered first. Boosting can suitably be measured by, for example, antibody titres, using methods standard in the art such as ELISA.

In one aspect an HPV L1 protein or immunogenic fragment thereof is used for boosting cross reactive antibodies previously raised to a different HPV type.

In one aspect the invention relates to use of L1 protein or immunogenic fragment thereof from one HPV type in boosting the immune response against a second, different HPV type.

In one aspect the invention relates to use of L1 protein or immunogenic fragment thereof from one HPV type in boosting the immune response against a homologous HPV type.

In one aspect the invention relates to use of L1 protein or immunogenic fragment thereof from one HPV type in boosting the immune response against a second, different HPV type, wherein the second type is phylogenetically related to the first type. Phylogenetic relationships between HPV types are well known in the art (see e.g. de Villers et al. Virology. 2004 June 20; 324(1):17-27). In this publication papillomaviruses can be seen to fall into distinct species which are phylogenetically related. For example, in species 16 are found types 16, 31, 33. In species 7 are found types 18 and 45.

In another aspect the invention relates to use of HPV 31 L1 protein or immunogenic fragment thereof in the preparation of a medicament for boosting an immune response to (generated by) an HPV 16 vaccine comprising an L1 protein or fragment thereof.

In another aspect the invention relates to use of HPV 52 L1 protein or immunogenic fragment thereof in the preparation of a medicament for boosting an immune response to an HPV 16 vaccine comprising an L1 protein or fragment thereof.

In another aspect the invention relates to use of HPV 45 L1 protein or immunogenic fragment thereof in the preparation of a medicament for boosting an immune response to an HPV 18 vaccine comprising an L1 protein or fragment thereof.

In another aspect the invention relates to use of HPV 18 L1 protein or immunogenic fragment thereof in the preparation of a medicament for boosting an immune response to an HPV 45 vaccine comprising an L1 protein or fragment thereof.

In another aspect the invention relates to use of HPV 16 L1 protein or immunogenic fragment thereof in the preparation of a medicament for boosting an immune response to an HPV 31 vaccine comprising an L1 protein or fragment thereof.

In another aspect the invention relates to use of HPV 16 L1 protein or immunogenic fragment thereof in the preparation of a medicament for boosting an immune response to an HPV 52 vaccine comprising an L1 protein or fragment thereof.

The present relates in one aspect to a vaccination schedule for protection against HPV infection or disease, the schedule comprising delivery of a first HPV vaccine comprising an L1 protein or immunogenic fragment thereof from at least HPV 16 and HPV 18, and after a suitable interval a second HPV vaccine which does not comprise the HPV 16 and HPV 18 L1 components from the first vaccine, and which second vaccine comprises an L1 protein or immunogenic fragment thereof from at least one other oncogenic HPV type. In this aspect of the invention the components of the first and second vaccines as defined above can be delivered in reverse order. By way of example, the first HPV to be delivered might comprise HPV 31 and HPV 45 antigens, while the second vaccine to be delivered comprises HPV 16 and HPV 18 antigens.

The vaccines of the invention can additionally comprise, within the constraints of the invention, antigens from other HPV types, such as other antigenic oncogenic types (e.g. HPV 31, 33, 35, 39, 45, 51, 52, 56, 58, 59, 66, 68.), skin types (e.g. types 5, 8) and genital warts types (6, 11).

Suitably each vaccine is capable of protection against persistent infection for HPV types present in the vaccine.

Suitably each vaccine is capable of protection against incident infection for HPV types present in the vaccine.

Incident and persistent cervical infection are defined in Example 1.

Suitably each vaccine is capable of protection against cytological abnormalities related to HPV infection (e.g. ASCUS, CIN 1, CIN2, CIN3, cervical cancer), suitably caused by types not present in the vaccine, such as HPV 31, 45 or 52. L1 proteins or protein fragments from additional HPV types can be included in the vaccine of the invention, such as skin types (in particular HPV 5 and 8) and types associated with genital warts, such as HPV 6 and 11. Types 6 and 11 are not considered oncogenic types herein.

In one aspect the vaccine can comprise HPV L1 protein components, preferably as virus like particles, in different amounts. In one aspect, HPV 16 and HPV 18 VLPs may be provided at a higher dose than other oncogenic types, such as HPV 33 or 58. In one aspect HPV 16 and HPV 18 L1 only VLPs are provided at 20 μg per dose for human use. Other HPV VLPs may be used at a lower dose, such as 15 or 110 μg per dose for human use.

In one aspect of the invention the vaccine may include an HPV early antigen, for example an antigen selected from the list consisting of HPV E1, E2, E3, E4, E5, E6, E7 or E8. In an alternative aspect the vaccine may lack an HPV early antigen, for example an antigen selected from the list consisting of HPV E1, E2, E3, E4, E5, E6, E7 or E8.

In one aspect a vaccine component of the invention is trivalent (contains an HPV L1 or fragment thereof from 3 different oncogenic HPV types). In a further aspect the vaccine is tetravalent. In a further aspect the vaccine is pentavalent. In a further aspect the vaccine is hexavalent. In a further aspect the vaccine is heptavalent. In a further aspect the vaccine is octavalent. Higher order valancies are also contemplated herein. In further aspects the vaccine is at least tetravalent, or at least pentavalent, or at least hexavalent, or at least heptavalent or at least octavalent with respect to the oncogenic HPV types. In a further aspect the combined vaccines of the invention comprise between them L1 protein or immunogenic fragments thereof from 4, 5, 6, 7, 8, 9 or 10 different HPV types.

Preferably the combination of HPV components within the vaccine does not significantly impact the immunogenicity of any one HPV component. In particular it is preferred that there is no biologically relevant interference between HPV antigens in the combination of the invention, such that the combined vaccine of the invention is able to offer effective protection against infection by each HPV genotype represented in the vaccine. Suitably the immune response against a given HPV type in the combination is at least 50% of the immune response of that same HPV type when measured individually, preferably 100% or substantially 100%. For responses to the HPV 16 and HPV 18, the combined vaccine of the invention preferably stimulates an immune response which is at least 50% of that provided by a combined HPV 16/HPV 18 vaccine. Suitably the immune response generated by the vaccine of the invention is at a level in which the protective effect of each HPV type is still seen. The immune response may suitably be measured, for example, by antibody responses, in either preclinical or human experiments. Measurement of antibody responses is well known in the art, and disclosed in (for example) WO03/077942.

Where the vaccine or composition of the invention comprises an immunogenic fragment of L1, then suitable immunogenic fragments of HPV L1 include truncations, deletions, substitution, or insertion mutants of L1. Such immunogenic fragments are suitably capable of raising an immune response (if necessary, when adjuvanted), said immune response being capable of recognising an L1 protein such as a virus like particle, from the HPV type from which the L1 protein was derived.

In one aspect a suitable immunogenic fragment of HPV 16 is capable of cross protection against at least one of HPV 31 and HPV 52, and in an aspect of the invention, capable of cross protection against both.

In another aspect a suitable immunogenic fragment of HPV 18 is capable of cross protection against HPV 45.

Cross protection obtainable by immunogenic fragments of HPV 16 and/or HPV 18 can be assessed by trials in humans, for example as outlined in Example 1.

Similarly, different vaccines according to the present invention can be tested using standard techniques, for example as in Example 1, or in standard preclinical models, to confirm that the vaccine is immunogenic.

Suitable immunogenic L1 fragments include truncated L1 proteins. In one aspect the truncation removes a nuclear localisation signal. In another aspect the truncation is a C terminal truncation. In a further aspect the C terminal truncation removes fewer than 50 amino acids, such as fewer than 40 amino acids. Where the L1 is from HPV 16 then in another aspect the C terminal truncation removes 34 amino acids from HPV 16 L1. Where the L1 is from HPV 18 then in a further aspect the C terminal truncation removes 35 amino acids from HPV 18 L1.

| In one aspect the HPV 16 sequence is the following sequence: | |
|---|---|
| (SEQ ID NO: 1) | |
| MSLWLPSEATVYLPPVPVSKVVSTDEYVARTNIYYHAGTSRLLAVGHPYFPIKKPNNNKI | 60 |
| LVPKVSGLQYRVFRIHLPDPNKFGFPDTSFYNPDTQRLVWACVGVEVGRGQPLGVGISGH | 120 |
| PLLNKLDDTENASAYAANAGVDNRECISMDYKQTQLCLIGCKPPIGEHWGKGSPCTNVAV | 180 |
| NPGDCPPLELINTVIQDGDMVDTGFGAMDFTTLQANKSEVPLDICTSICKYPDYIKMVSE | 240 |
| PYGDSLFFYLRREQMFVRHLFNRAGAVGENVPDDLYIKGSGSTANLASSNYFPTPSGSMV | 300 |
| TSDAQIFNKPYWLQRAQGHNNGICWGNQLFVTVVDTTRSTNMSLCAAISTSETTYKNTNF | 360 |
| KEYLRHGEEYDLQFIFQLCKITLTADVMTYIHSMNSTILEDWNFGLQPPPGGTLEDTYRF | 420 |
| VTSQAIACQKHTPPAPKEDPLKKYTFWEVNLKEKFSADLDQFPLGRKFLLQ | 471 |

In another aspect the invention relates to virus like particles consisting only of HPV 16 L1 having the amino sequence above, and to compositions containing such VLPs.

The HPV 16 sequence may also be that disclosed in WO9405792 or U.S. Pat. No. 6,649,167, for example, suitably truncated. Suitable truncates are truncated at a position equivalent to that shown above, as assessed by sequence comparison.

| In one aspect the HPV 18 sequence is the following sequence: | |
|---|---|
| (SEQ ID NO: 2) | |
| MALWRPSDNTVYLPPPSVARVVNTDDYVTRTSIFYHAGSSRLLTVGNPYFRVPAGGGNKQ | 60 |
| DIPKVSAYQYRVFRVQLPDPNKFGLPDNSIYNPETQRLVWACVGVEIGRGQPLGVGLSGH | 120 |
| PFYNKLDDTESSHAATSNVSEDVRDNVSVDYKQTQLCILGCAPAIGEHWAKGTACKSRPL | 180 |
| SQGDCPPLELKNTVLEDGDMVDTGYGAMDFSTLQDTKCEVPLDICQSICKYPDYLQMSAD | 240 |
| PYGDSMFFCLRREQLFARHFWNRAGTMGDTVPPSLYIKGTGMRASPGSCVYSPSPSGSIV | 300 |
| TSDSQLFNKPYWLHKAQGHNNGVCWHNQLFVTVVDTTRSTNLTICASTQSPVPGQYDATK | 360 |
| FKQYSRHVEEYDLQFIFQLCTITLTADVMSYIHSMNSSILEDWNFGVPPPPTTSLVDTYR | 420 |
| FVQSVAITCQKDAAPAENKDPYDKLKFWNVDLKEKFSLDLDQYPLGRKFLVQ | 472 |

In another aspect the invention relates to virus like particles consisting only of HPV 18 L1 having the amino sequence above, and to compositions containing such VLPs.

An alternative HPV 18 sequence is disclosed in WO9629413, which may be suitably truncated. Suitable truncates are truncated at a position equivalent to that shown above, as assessed by sequence comparison.

Other HPV 16 and HPV 18 sequences are well known in the art and may be suitable for use in the present invention.

Where the L1 protein is from another HPV type then C terminal truncations corresponding to those made for HPV 16 and HPV 18 may be used, based upon DNA or protein sequence alignments.

Suitable truncations of, for example, HPV 31, HPV 45, HPV 52, HPV 58, HPV 33 may also be made, in one aspect removing equivalent C terminal portions of the L1 protein to those described above, as assessed by sequence alignment.

| Nucleotidic Sequence of Truncated L1 from serotype 31 | |
|---|---|
| (SEQ ID NO: 5) | |
| ATGTCTCTGTGGCGGCCTAGCGAGGCTACTGTCTACTTACCACCTGTCCCAGTGTCTAAAGTTGTAAGCA | 70 |
| CGGATGAATATGTAACACGAACCAACATATATTATCACGCAGGCAGTGCTAGGCTGCTTACAGTAGGCCA | 140 |
| TCCATATTATTCCATACCTAAATCTGACAATCCTAAAAAAATAGTTGTACCAAAGGTGTCAGGATTACAA | 210 |
| TATAGGGTATTTAGGGTTCGTTTACCAGATCCAAACAAATTTGGATTTCCTGATACATCTTTTTATAATC | 280 |
| CTGAAACTCAACGCTTAGTTTGGGCCTGTGTTGGTTTAGAGGTAGGTCGCGGGCAGCCATTAGGTGTAGG | 350 |
| TATTAGTGGTCATCCATTATTAAATAAATTTGATGACACTGAAAACTCTAATAGATATGCCGGTGGTCCT | 420 |
| GGCACTGATAATAGGGAATGTATATCAATGGATTATAAACAAACACAACTGTGTTTACTTGGTTGCAAAC | 490 |
| CACCTATTGGAGAGCATTGGGGTAAAGGTAGTCCTTGTAGTAACAATGCTATTACCCCTGGTGATTGTCC | 560 |
| TCCATTAGAATTAAAAAATTCAGTTATACAAGATGGGGATATGGTTGATACAGGCTTTGGAGCTATGGAT | 630 |
| TTTACTGCTTTACAAGACACTAAAAGTAATGTTCCTTTGGACATTTGTAATTCTATTTGTAAATATCCAG | 700 |
| ATTATCTTAAAATGGTTGCTGAGCCATATGGCGATACATTATTTTTTTATTTACGTAGGGAACAAATGTT | 770 |
| TGTAAGGCATTTTTTTAATAGATCAGGCACGGTTGGTGAATCGGTCCCTACTGACTTATATATTAAAGGC | 840 |
| TCCGGTTCAACAGCTACTTTAGCTAACAGTACATACTTTCCTACACCTAGCGGCTCCATGGTTACTTCAG | 910 |
| ATGCACAAATTTTTAATAAACCATATTGGATGCAACGTGCTCAGGGACACAATAATGGTATTTGTTGGGG | 980 |
| CAATCAGTTATTTGTTACTGTGGTAGATACCACACGTAGTACCAATATGTCTGTTTGTGCTGCAATTGCA | 1050 |
| AACAGTGATACTACATTTAAAAGTAGTAATTTTAAAGAGTATTTAAGACATGGTGAGGAATTTGATTTAC | 1120 |
| AATTTATATTTCAGTTATGCAAAATAACATTATCTGCAGACATAATGACATATATTCACAGTATGAATCC | 1190 |
| TGCTATTTTGGAAGATTGGAATTTTGGATTGACCACACCTCCCTCAGGTTCTTTGGAGGATACCTATAGG | 1260 |
| TTTGTCACCTCACAGGCCATTACATGTCAAAAAACTGCCCCCCAAAAGCCCAAGGAAGATCCATTTAAAG | 1330 |
| ATTATGTATTTTGGGAGGTTAATTTAAAAGAAAAGTTTTCTGCAGATTTAGATCAGTTTCCACTGGGTCG | 1400 |
| CAAATTTTTATTACAGTAA | 1419 |

In another aspect the invention relates to virus like particles consisting only of HPV 31 L1 having the amino sequence encoded by the sequence above, and to compositions containing such VLPs.

| Nucleotidic Sequence of Truncated L1 from serotype 45 | |
|---|---|
| (SEQ ID NO: 6) | |
| ATGGCTTTGTGGCGGCCTAGTGACAGTACGGTATATCTTCCACCACCTTCTGTGGCCAGAGTTGTCAACA | 70 |
| CTGATGATTATGTGTCTCGCACAAGCATATTTTACCATGCAGGCAGTTCCCGATTATTAACTGTAGGCAA | 140 |
| TCCATATTTTAGGGTTGTACCTAGTGGTGCAGGTAATAAACAGGCTGTTCCTAAGGTATCCGCATATCAG | 210 |
| TATAGGGTGTTTAGAGTAGCTTTACCCGATCCTAATAAATTTGGATTACCTGATTCTACTATATATAATC | 280 |
| CTGAAACACAACGTTTGGTTTGGGCATGTGTAGGTATGGAAATTGGTCGTGGGCAGCCTTTAGGTATTGG | 350 |
| CCTAAGTGGCCATCCATTTTATAATAAATTGGATGATACAGAAAGTGCTCATGCGGCTACGGCTGTTGTT | 420 |
| ACGCAGGATGTTAGGGATAATGTGTCAGTTGATTATAAGCAAACACAGCTGTGTATTTTAGGTTGTGTAC | 490 |
| CTGCTATTGGTGAGCACTGGGCCAAGGGCACACTTTGTAAACCTGCACAATTGCAGCCTGGTGACTGTCC | 560 |
| TCCTTTGGAACTTAAAAACACCATTATTGAGGATGGTGATATGGTGGATACAGGTTATGGGGCAATGGAT | 630 |
| TTTAGTACATTGCAGGATACAAAGTGCGAGGTTCCATTAGACATTTGTCAATCCATCTGTAAATATCCAG | 700 |
| ATTATTTGCAAATGTCTGCTGATCCCTATGGGGATTCTATGTTTTTTTGCCTACGCCGTGAACAACTGTT | 770 |
| TGCAAGACATTTTTGGAATAGGGCAGGTGTTATGGGTGACACAGTACCTACAGACCTATATATTAAAGGC | 840 |
| ACTACCGCTAATATGCGTGAAACCCCTGGCAGTTGTGTGTATTCCCCTTCTCCCAGTGGCTCTATTATTA | 910 |
| CTTCTGATTCTCAATTATTTAATAAGCCATATTGGTTACATAAGGCCCAGGGCCATAACAATGGTATTTG | 980 |
| TTGGCATAATCAGTTGTTTGTTACTGTAGTGGACACTACCCGCAGTACTAATTTAACATTATGTGCCTCT | 1050 |
| ACACAAAATCCTGTGCCAGGTACATATGATCCTACTAAGTTTAAGCACTATAGTAGACATGTGGAGGAAT | 1120 |
| ATGATTTACAGTTTATTTTTCAGTTGTGCACTATTACTTTAACTGCAGAGGTTATGTCATATATCCATAG | 1190 |
| TATGAATAGTAGTATATTGGAAAATTGGAATTTTGGTGTACCTCCACCACCTACTACAAGTTTAGTGGAT | 1260 |
| ACATATCGTTTTGTGCAATCAGTTGCTGTTACCTGTCAAAAGGATACTACACCTCCAGAAAAGCAGGATC | 1330 |
| CATATGATAAATTAAAGTTTTGGACTGTTGACCTAAAGGAAAAATTTTCCTCCGATTTGGATCAATATCC | 1400 |
| CCTTGGTCGAAAGTTTTTAGTTCAGTAA | |

In another aspect the invention relates to virus like particles consisting only of HPV 45 L1 having the amino sequence encoded by the sequence above, and to compositions containing such VLPs.

The L1 protein or fragment of the invention may optionally be in the form of a fusion protein, such as the fusion of the L1 protein with L2 or an early protein.

The HPV L1 protein is suitably in the form of a capsomer or virus like particle (VLP). In one aspect HPV VLPs may be used in the present invention. HPV VLPs and methods for the production of VLPs are well known in the art. VLPs typically are constructed from the L1 and optionally L2 structural proteins of the virus, see for example WO9420137, U.S. Pat. No. 5,985,610, WO9611272, U.S. Pat. No. 6,599,508B1, U.S. Pat. No. 6,361,778B1, EP 595935. Any suitable HPV VLP may be used in the present invention which provides cross protection, such as an L1 or L1+L2 VLP.

Suitably the VLP is an L1-only VLP.

VLP formation can be assessed by standard techniques such as, for example, electron microscopy and dynamic laser light scattering.

The VLP may comprise full length L1 protein. In one aspect the L1 protein used to form the VLP is a truncated L1 protein, as described above.

VLPs may be made in any suitable cell substrate such as yeast cells or insect cells e.g. baculovirus cells, and techniques for preparation of VLPs are well known in the art, such as WO9913056, U.S. Pat. No. 6,416,945B1, U.S. Pat. No. 6,261,765B1 and U.S. Pat. No. 6,245,568, and references therein, the entire contents of which are hereby incorporated by reference.

VLPS are suitably made by disassembly and reassembly techniques, which can provide for more stable and/or homogeneous papillomavirus VLPs. For example, McCarthy et al, 1998 “Quantitative Disassembly and Reassembly of Human Papillomavirus Type 11 Virus like Particles in Vitro” J. Virology 72(1):33-41, describes the disassembly and reassembly of recombinant L1 HPV 11 VLPs purified from insect cells in order to obtain a homogeneous preparation of VLP's. WO9913056 and U.S. Pat. No. 6,245,568 also describe disassembly/reassembly processes for making HPV VLPs.

In one aspect HPV VLPS are made as described WO9913056 or U.S. Pat. No. 6,245,568

The HPV L1 the invention may be combined with an adjuvant or immunostimulant such as, but not limited to, detoxified lipid A from any source and non-toxic derivatives of lipid A, saponins and other reagents capable of stimulating a TH1 type response.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p407-419) and has the following structure:

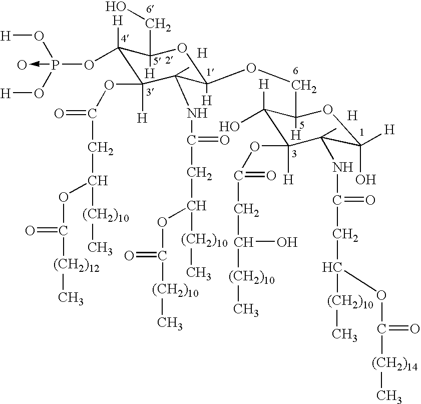

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof.

A suitable form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO9843670A2.

The bacterial lipopolysaccharide derived adjuvants to be formulated in the compositions of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al., 1986, Int. Arch. Allergy. Immunol., 79(4):392-6; Hilgers et al., 1987, Immunology, 60(1):141-6; and EP 0 549 074 B1). In one aspect the bacterial lipopolysaccharide adjuvant is 3D-MPL.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Other saponins which have been used in systemic vaccination studies include those derived from other plant species such as Gypsophila and Saponaria (Bomford et al., Vaccine, 10(9):572-577, 1992).

An enhanced system involves the combination of a non-toxic lipid A derivative and a saponin derivative particularly the combination of QS21 and 3D-MPL as disclosed in WO 94/00153, or a less reactogenic composition where the QS21 is quenched with cholesterol as disclosed in WO 96/33739.

A particularly potent adjuvant formulation involving QS21 and 3D-MPL in an oil in water emulsion is described in WO 95/17210 and use of this adjuvant forms an aspect of the invention.

Accordingly in one embodiment of the present invention there is provided a vaccine adjuvanted with detoxified lipid A or a non-toxic derivative of lipid A, more suitably adjuvanted with a monophosphoryl lipid A or derivative thereof.

In one aspect the vaccine additionally comprises a saponin, for example QS21.

In one aspect the formulation additionally comprises an oil in water emulsion. The present invention also provides a method for producing a vaccine formulation comprising mixing an L2 peptide of the present invention together with a pharmaceutically acceptable excipient, such as 3D-MPL.

Additional components that may be included present in an vaccine formulation according to the invention include non-ionic detergents such as the octoxynols and polyoxyethylene esters as described herein, particularly t-octylphenoxy polyethoxyethanol (Triton X-100) and polyoxyethylene sorbitan monooleate (Tween 80); and bile salts or cholic acid derivatives as described herein, in particular sodium deoxycholate or taurodeoxycholate. Thus, in one aspect of the invention a formulation comprises 3D-MPL, Triton X-100, Tween 80 and sodium deoxycholate, which may be combined with an L2 antigen preparation to provide a suitable vaccine.

In one embodiment of the present invention, the vaccine comprises a vesicular adjuvant formulation comprising cholesterol, a saponin and an LPS derivative. In this regard the adjuvant formulation suitably comprises a unilamellar vesicle comprising cholesterol, having a lipid bilayer suitably comprising dioleoyl phosphatidyl choline, wherein the saponin and the LPS derivative are associated with, or embedded within, the lipid bilayer. In one aspect these adjuvant formulations comprise QS21 as the saponin, and 3D-MPL as the LPS derivative, wherein the ratio of QS21:cholesterol is from 1:1 to 1:100 weight/weight, and in one aspect, a ratio of 1:5 weight/weight. Such adjuvant formulations are described in EP 0 822 831 B, the disclosure of which is incorporated herein by reference.

Suitably the vaccines of the invention are used in combination with aluminium, and are suitably adsorbed or partially adsorbed onto aluminium adjuvants. Suitably the adjuvant is an aluminium salt, which may be in combination with 3D MPL, such as aluminium phosphate and 3D MPL. Aluminium hydroxide, optionally in combination with 3D MPL is also suitable.

In another aspect of the present invention the vaccine comprises the combination of HPV VLPs with an aluminium salt or with an aluminium salt +3D MPL. Aluminium hydroxide is suitable as the aluminium salt.

The vaccine may also comprise aluminium or an aluminium compound as a stabiliser.

In another aspect the adjuvant may be a combination of an oil-in-water emulsion adjuvant and 3D MPL. In one aspect the oil-in-water emulsion comprises a metabolisable oil, a sterol and an emulsifying agent.

The vaccines of the invention may be provided by any of a variety of routes such as oral delivery (e.g. see WO9961052 A2), topical, subcutaneous, mucosal (typically intravaginal), intravenous, intramuscular, intranasal, sublingual, intradermal and via suppository.

Optionally the vaccine may also be formulated or co-administered with other HPV antigens or non-HPV antigens. Suitably these non-HPV antigens can provide protection against other diseases, such as sexually transmitted diseases such as herpes simplex virus, EBV, chlamydia and HIV. We particularly prefer that the vaccine comprises gD or a truncate thereof from HSV. In this way the vaccine provides protection against both HPV and HSV.

The dosage of the vaccine components will vary with the condition, sex, age and weight of the individual, the administration route and HPV of the vaccine. The quantity may also be varied with the number of VLP types. Suitably the delivery is of an amount of vaccine suitable to generate an immunologically protective response. Suitably each vaccine dose comprises 1-100 μg of each VLP, in one aspect 5-80 μg, in another aspect 5-30 μg each VLP, in a further aspect 5-20 μg of each VLP, in a yet further aspect 5 μg, 6 μg, 10 μg, 15 μg or 20 μg.

For all vaccines of the invention, in one aspect the vaccine is used for the vaccination of adolescent girls aged 10-15, such as 10-13 years. However, older girls above 15 years old and adult women may also be vaccinated. The vaccine may also be administered to women following an abnormal pap smear or after surgery following removal of a lesion caused by HPV, or who are seronegative and DNA negative for HPV cancer types.

In one aspect the vaccine of the invention is used to vaccinate males.

In one aspect the vaccine is delivered in a 2 dose regime, for example in a 0, 1 month regime or 0, 6 month regime respectively. The vaccination regime may incorporate a booster injection after 5 to 10 years, such as 10 years.

In one aspect the invention relates to a three dose vaccine, in which (for example) a first vaccine and second vaccine are delivered, the first and second vaccine being different, followed by a third vaccine containing one or more or all of the HPV elements of the first or second vaccines. In one aspect the first and second vaccines are completely different with respect to the HPV L1 components.

For example, a first vaccine may comprise or consist of HPV 16 and HPV 18 L1 protein. The second vaccine may consist of HPV 31 and HPV 45 L1 protein. A third vaccine may comprise L1 protein from all 4 HPV types, HPV 16, 18, 31 and 45.

In another aspect the invention relates to a three dose vaccine, in which (for example) a first HPV vaccine and second HPV vaccine are delivered, the first and second vaccine being different, followed by a third vaccine, the third vaccine containing none of the HPV L1 components of the first or second vaccines.

In one aspect the vaccine is a liquid vaccine formulation, although the vaccine may be lyophilised and reconstituted prior to administration.

The teaching of all references in the present application, including patent applications and granted patents, are herein fully incorporated by reference.

The vaccines of the invention comprise certain HPV components as laid out above. In a further aspect of the invention the vaccine consists essentially of, or consists of, said components.

The term ‘vaccine’, as used in the present invention, refers to a composition that comprises an immunogenic component capable of provoking an immune response in an individual, such as a human, optionally when suitably formulated or adjuvanted. A vaccine suitably elicts a protective immune response against incident infection, or persistent infection, or cytological abnormality such as ASCUS, CIN1, CIN2 CIN3, or cervical cancer caused by one or more HPV types.

The present invention is now described with respect to the following examples which serve to illustrate the invention.

### Example 1

Precise details of the experiment carried out are provided in Harper et al, the Lancet. 2004 Nov. 13; 364(9447):1757-65.

In summary, healthy women between the ages of 15 and 25 years were immunised with a mixture of HPV 16 and HPV 18 L1 VLPs. The women at enrolment were: 1) seronegative for HPV-16 and HPV-18; 2) negative for high risk HPV infection of the cervix (detected by HPV PCR); 3) had 6 or fewer lifetime sexual partners and 4) had normal PAP smears.

The mixture comprised, per 0.5 ml dose, 20 μg of HPV-16 L1 VLP, 20 μg of HPV-18 L1 VLP and was adjuvanted with 500 μg of aluminum hydroxide and 50 μg of 3D MPL. The placebo group was injected with 500 μg of aluminum hydroxide alone.

The vaccine efficacy (V.E.) against certain cancer HPV types was assessed, wherein the V.E. is the % improvement in protection against infection or disease by the vaccine compared to a placebo group.

Cross protection was assessed by detecting the presence of nucleic acid specific for various oncogenic types in the vaccinees and control group. Detection was carried out using techniques as described in WO03014402, and references therein, particularly for non-specific amplification of HPV DNA and subsequent detection of DNA types using a LiPA system as described in WO 99/14377, and in Kleter et al, [Journal of Clinical Microbiology (1999), 37 (8): 2508-2517], the whole contents of which are herein specifically incorporated by reference.

Any suitable method can, however, be used for the detection of HPV DNA in a sample, such as type specific PCR using primers specific for each HPV type of interest. Suitable primers are known to the skilled person, or can be easily constructed given that the sequences of the oncogenic HPV types are known.

In detail, the methods section of the Lancet paper is reproduced here, for completeness:

The primary objective of this study was to assess vaccine efficacy in the prevention of infection with HPV-16, HPV-18, or both (HPV-16/18), between months 6 and 18 in participants who were initially shown to be seronegative for HPV-16/18 by ELISA and negative for HPV-16/18 DNA by PCR. Secondary objectives included: evaluation of vaccine efficacy in the prevention of persistent infection with HPV-16/18, and the evaluation of vaccine efficacy in the prevention of cytologically confirmed low-grade squamous intraepithelial lesions (LSIL), high-grade squamous intraepithelial lesions (HSIL), and histologically confirmed LSIL (CIN 1), HSIL (CIN 2 or 3) squamous cell cancer, or adenocarcinoma associated with HPV-16/18 infection between months 6 and 18, and months 6 and 27. The prevention of atypical squamous cells of undetermined significance (ASCUS) cytology associated with HPV-16/18 infection was added post-hoc to the outcome analyses.

We also did an exploratory analysis of the histopathological endpoints CIN 1 and 2 associated with HPV-16/18 DNA detected by PCR in lesional tissue. Other objectives included the assessment of vaccine immunogenicity, safety, and tolerability.

Investigators in North America (Canada and the USA) and Brazil recruited women for this efficacy study through advertisements or previous participation in an HPV cross-sectional epidemiology study that took place between July and December, 2000.

For each of the 32 study sites, an institutional review board approved the protocol, consent forms, and amendments. Women signed separate written consents for study participation and colposcopy. For those under 18 years, parental consent and assent from the participant were obligatory.

There were two study phases: an initial phase for vaccination and follow-up that concluded at month 18; and a blinded follow-up extension phase that concluded at month 27.

Women eligible for the initial phase (months 0-18) included healthy women aged 15-25 years, who had had no more than six sexual partners, no history of an abnormal Pap test or ablative or excisional treatment of the cervix, and no ongoing treatment for external condylomata; and who were cytologically negative, seronegative for HPV-16 and HPV-18 antibodies by ELISA, and HPV-DNA-negative by PCR for 14 high-risk HPV types (16, 18, 31, 33, 35, 39, 45, 51, 52, 56, 58, 59, 66, and 68) no more than 90 days before study entry.

Women who completed the initial phase of the study earliest, and who did not have ablative or excisional therapy of the cervix, or hysterectomy after enrolment, were eligible to participate in the extension phase of the study (months 18-27).

### Procedures

Each dose of the bivalent HPV-16/18 virus-like particle vaccine (GlaxoSmithKline Biologicals, Rixensart, Belgium) contained 20 μg of HPV-16 L1 virus-like particle and 20 μg of HPV-18 L1 virus-like particle. Each type of virus-like particle was produced on Spodoptera frugiperda Sf-9 and Trichoplusia ni Hi-5 cell substrate with AS04 adjuvant containing 500 μg aluminum hydroxide and 50 μg 3-deacylated monophosphoryl lipid A (MPL, Corixa, Mont., USA) provided in a monodose vial. The placebo contained 500 μg of aluminum hydroxide per dose, and was identical in appearance to the HPV-16/18 vaccine. Every study participant received a 0·5 mL dose of vaccine or placebo at 0 months, 1 month, and 6 months.

Health-care providers obtained cervical specimens with a cervical brush and spatula (washed in PreservCyt, Cytyc Corporation, Boxborough, Mass., USA) for cytology and HPV DNA testing at screening and months 6, 12, and 18. At months 0 and 6, and subsequently every 3 months, women self-obtained cervicovaginal samples with two sequential swabs (placed in PreservCyt) for HPV DNA testing. [D M Harper, W W Noll, D R Belloni and B F. Cole, Randomized clinical trial of PCR-determined human papillomavirus detection methods: self-sampling versus clinician-directed-biologic concordance and women's preferences. Am J Obstet Gynecol 186 (2002), pp. 365-373] A central laboratory (Quest Diagnostics, Teterboro, N.J., USA) reported cytology results (ThinPrep, Cytyc Corporation) by use of the 1991 Bethesda classification system.

Protocol guidelines recommended colposcopy after two reports of ASCUS, or one report of atypical glandular cells of undetermined significance, LSIL or HSIL, squamous cell carcinoma, adenocarcinoma in situ, or adenocarcinoma. These guidelines also recommended biopsy for any suspected lesions.

The central histology laboratory made an initial diagnosis from the formalin-fixed tissue specimens for clinical management. A panel of three pathologists made a subsequent consensus diagnosis for HPV-16 and HPV-18 associated lesions with the CIN system. This consensus diagnosis also included review of the sections taken at the time of microdissection for PCR detection of lesional HPV DNA.

HPV DNA isolated from the cytology specimen (MagNaPure Total Nucleic Acid system, Roche Diagnostics, Almere, Netherlands) and from the cervical biopsy specimen (proteinase K extraction) was amplified from an aliquot of purified total DNA with the SPF10 broad-spectrum primers that amplify a 65 bp region of the L1 gene. [B Kleter, L J van Doom, J ter Schegget et al., Novel short-fragment PCR assay for highly sensitive broad-spectrum detection of anogenital human papillomaviruses. Am J Pathol 153 (1998), pp. 1731-1739: L J van Doom, W Quint, B Kleter et al., Genotyping of human papillomavirus in liquid cytology cervical specimens by the PGMY line blot assay and the SPF(10) line probe assay. J Clin Microbiol 40 (2002), pp. 979-983 and W G Quint, G Scholte, L J van Doom, B Kleter, P H Smits and J. Lindeman, Comparative analysis of human papillomavirus infections in cervical scrapes and biopsy specimens by general SPF(10) PCR and HPV genotyping. J Pathol 194 (2001), pp. 51-58] The amplification products were detected by a DNA enzyme immunoassay. A line probe assay (LiPA Kit HPV INNO LiPA HPV genotyping assay, SPF-10 system version 1, Innogenetics, Gent, Belgium, manufactured by Labo Bio-medical Products, Rijswijk, Netherlands) detected 25 HPV genotypes (6, 11, 16, 18, 31, 33, 34, 35, 39, 40, 42, 43, 44, 45, 51, 52, 53, 56, 58, 59, 66, 68, 70, and 74). [B Kleter, L J van Doom, L Schrauwen et al., Development and clinical evaluation of a highly sensitive PCR-reverse hybridization line probe assay for detection and identification of anogenital human papillomavirus. J Clin Microbiol 37 (1999), pp. 2508-2517] Any specimen that was positive by DNA enzyme immunoassay was tested by type-specific HPV-16 and HPV-18 PCR. HPV-16 type-specific PCR primers amplified a 92 bp segment of the E6/E7 gene and HPV-18 type-specific PCR primers amplified a 126 bp segment of the L1 gene. [M F Baay, W G Quint, J Koudstaal et al, Comprehensive study of several general and type-specific primer pairs for detection of human papillomavirus DNA by PCR in paraffin-embedded cervical carcinomas. J Clin Microbiol 34 (1996), pp. 745-747]

We defined incident cervical infection with HPV-16/18 as at least one positive PCR result for HPV-16 or HPV-18 during the trial, and persistent infection with HPV-16/18 as at least two positive HPV-DNA PCR assays for the same viral genotype separated by at least 6 months. [H Richardson, G Kelsall, P Tellier et al., The natural history of type-specific human papillomavirus infections in female university students. Cancer Epidemiol Biomarkers Prev 12 (2003), pp. 485-490 and A B Moscicki, J H Ellenberg, S Farhat and J. Xu, Persistence of human papillomavirus infection in HIV-infected and -uninfected adolescent girls: risk factors and differences, by phylogenetic type. J Infect Dis 190 (2004), pp. 3745] HPV-DNA test results were concealed from investigators during the study and cytological and histological diagnoses were only revealed for clinical management purposes. Analyses included HPV-16/18 DNA results for cervical specimens and combined cervical and self-obtained cervicovaginal specimens.

We collected serum from study participants at months 0, 1, 6, 7, 12, and 18 for assessment of immunogenicity. Serological testing for antibodies to HPV-16 and HPV-18 virus-like particles was by ELISA. Recombinant HPV-16 or HPV-18 virus-like particles were used as coating antigens for antibody detection (see webappendix http://image.thelancet.com/extras/04art10103webappendix.pdf). Seropositivity was defined as a titre greater than or equal to the assay cut-off titre established at 8 ELISA units/mL for HPV-16 and 7 ELISA units/mL for HPV-18. Typical natural titres were determined by use of blood samples obtained from women in the preceding epidemiology study who were found to be seropositive for HPV-16 or HPV-18 by ELISA.

Women recorded symptoms experienced during the first 7 days after vaccination on diary cards with a three-grade scale of symptom intensity. Additionally, they reported to study personnel by interview all adverse events within the first 30 days after vaccination. Information on serious adverse events and pregnancies was collected throughout the study.

### Statistical Methods

Assuming a 6% cumulative incidence rate of both HPV-16 and HPV-18 type infections over 12 months, we estimated that 500 women per treatment group would provide 80% power to assess a lower limit of the 95% CI of the vaccine efficacy above zero. We assumed an 80% retention rate over 18 months. Interim analyses for efficacy, safety, and immunogenicity were done for future study planning purposes only; the O'Brien and Fleming method was used to adjust the αvalue for the final analysis after interim analyses occurred (overall α=0·05; two-sided test). [P C O'Brien and T R. Fleming, A multiple testing procedure for clinical trials. Biometrics 35 (1979), pp. 549-556]

Stratified, block randomisation according to validated algorithms was centralised with an internet randomisation system. Stratification was according to age (15-17, 18-21, and 22-25 years) and region (North America and Brazil). Each vaccine dose was attributed a randomly chosen number based on specific participant information entered into the computerised randomisation system by study personnel. Treatment allocation remains concealed from investigators and the women participating in a long-term follow-up study.

The intention-to-treat and according-to-protocol cohorts are shown in the figure, in which the reasons for exclusion from analyses are listed in rank order; women who met more than one exclusion criterion were only counted once according to the highest ranking criterion. We refer to the sets of participants entered in the intention-to-treat and according-to-protocol analyses as cohorts, although the information used to restrict subject inclusion in the according-to-protocol was only known after follow-up.

We did both according-to-protocol and intention-to-treat analyses for efficacy. Calculation of vaccine efficacy in the according-to-protocol 18-month analysis was based on the proportion of participants with HPV-16/18 infection in the vaccinated versus placebo groups. Vaccine efficacy was defined as 1 minus the ratio between these two proportions; 95% CIs measured the precision of the efficacy estimates. p values were calculated with the two-sided Fisher's exact test. Corresponding rates were expressed as the numbers of cases with the outcome divided by the numbers of participants at risk. The according-to-protocol 18-month cohort included enrolled women who received three scheduled doses of vaccine and complied with the protocol as described in the figure.

Calculation of vaccine efficacy in the intention-to-treat and according-to-protocol 27-month analyses was based on the Cox proportional hazard model using the time-to-occurrence of cases with HPV-16/18 infection in the vaccinated versus placebo groups. This allowed controlling for the accrued person-time data in each group. Vaccine efficacy was calculated using 1 minus the hazard ratio and p values calculated using the log rank test. Corresponding rates were expressed as the number of cases divided by the total person-time. All enrolled women who received at least one dose of vaccine or placebo, were negative for high-risk HPV-DNA at month 0, and had any data available for outcome measurement were included in the intention-to-treat cohort. The according-to-protocol 27-month cohort included outcome results from the according-to-protocol 18-month cohort and results that occurred during the extension phase (from 18 months to 27 months).

Calculation of p values for the safety analysis was performed using Fisher's exact test comparisons. The cohort for safety analysis included all enrolled women who received at least one dose of vaccine or placebo and complied with specified, minimal protocol requirements (see figure below:)

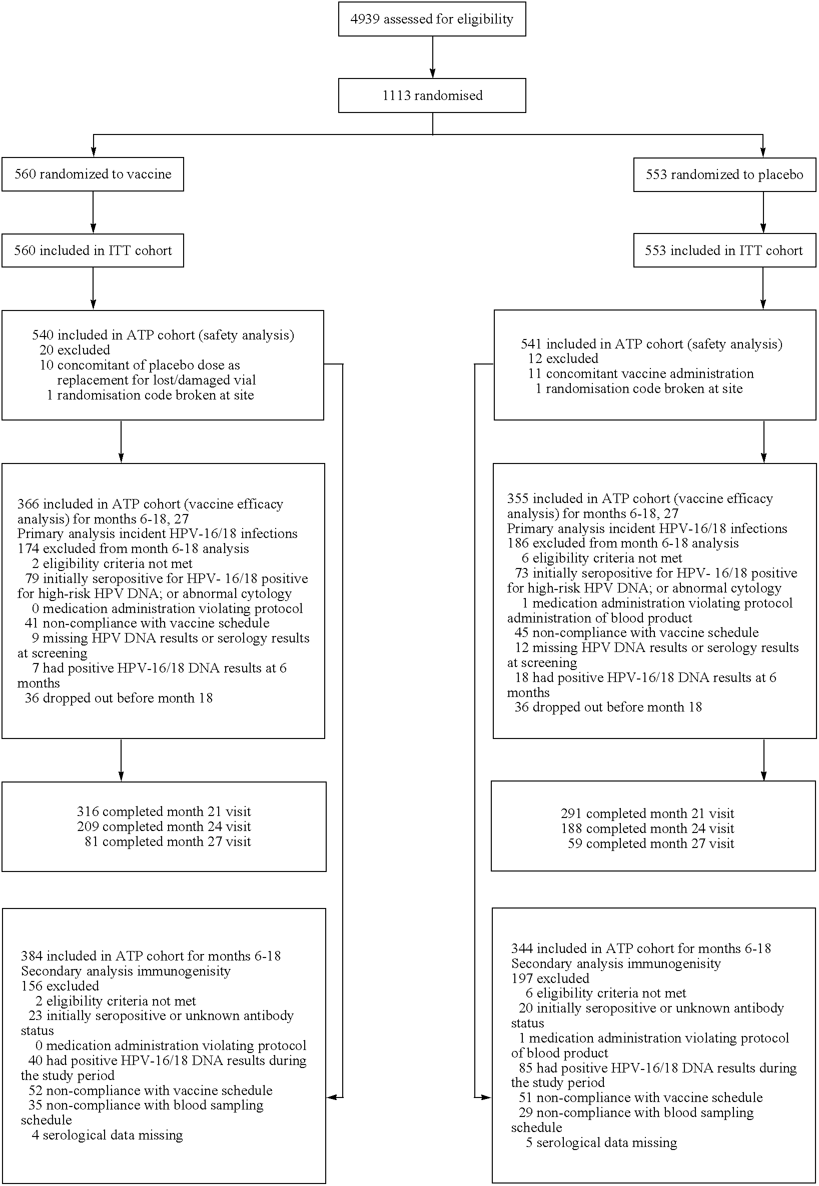

Immunogenicity was assessed in a subset of the according-to-protocol safety cohort, which included women with serology results at months 0, 7, and 18, who received all three doses of study vaccine or placebo according to schedule, complied with the blood sampling schedule, and did not become positive for HPV-16/18-DNA during the trial. Seropositivity rates between the vaccine and placebo groups were compared with Fisher's exact test (p<0·001 judged significant). Geometric mean titres were compared with ANOVA and Kruskal-Wallis test.

Block randomisation and statistical analyses were done with SAS version 8.2 (SAS Institute, Cary, N.C.).

### Initial Analysis and Results

Results of the initial analysis on cross protection are presented in patent application WO2004/056389, the whole contents of which herein incorporated by reference.

An initial analysis was carried out on an “ITT” (Intention To Treat cohort, representing all individuals who received at least one dose of vaccine). This data is shown in Table A.

The results presented in Tables B and C relate to the “ATP” (According To Protocol) group for those patients who complied with all the criteria of the trial. Table B is a midpoint analysis with data taken from all patients at the timepoint at which at least 50% of the cohort were 18 months after their first vaccination. Table C gives the final results, all data being from subjects at 18 months post first vaccination (month 0). In the ATP group all patients received 3 doses of vaccine at 0, 1 and 6 months and were seronegative at 6 months.

As demonstrated by the data presented in table A, immunization with a mixture of HPV16 and HPV18 VLPs provided apparent cross-protection against other HPV types. At this point the sample sizes are too small to provide for a rigorous statistical analysis, however the data demonstrate a positive trend and suggest that immunization with HPV 16 and HPV 18 VLPs will be efficacious against infection with other HPV types.

This was confirmed as the study progressed.

Table B demonstrates that HPV 16 and HPV 18 provide statistically significant cross protection against the group of high risk cancer types 31, 33, 35, 39, 45, 51, 52, 56, 58, 59, 66 and 68.

Table C demonstrates that, except for the HPV-18 related types (which show a very strong trend), there is statistically significant cross-protection against the groups of: HPV 31, 35, 58; HPV 31, 33, 35, 52, 58; and the 12 high risk (non HPV-16/18) types evaluated.

Further analysis was carried out on the specific cross protection against specific types.

Vaccine efficacy was assessed against infections and diseases related to the 12 high risk cancer types 31, 33, 35, 39, 45, 51, 52, 56, 58, 59, 66 and 68, HPV-16 phylogenetic-related types (the groups of; 31, 35, and 58; 31, 33, 35, 52 and 58) and HPV-18 phylogenetic related types (45 and 59).

An analysis was carried out on an “ATP” (According To Protocol) group for those patients who complied with all the criteria of the trial. In the ATP group all patients received 3 doses of vaccine at 0, 1 and 6 months and were seronegative at 6 months.

### Results

As demonstrated by the data presented in Table 1, immunization with a mixture of HPV16 and HPV18 VLPs provided statistically significant cross protection against incident infection by HPV types 31, 52 and 45 compared to the control.

Statistically significant cross protection against incident infection was also observed against the group of all HPV 16 related types (HPV-31, 33, 35, 52 and 58) and the group of all high risk types, excluding 16 and 18 (HPV 31, 33, 35, 39, 45, 51, 52, 56, 58, 59, 66, and 68).

Statistically significant cross protection against persistent infection was also observed against types 31 and 52 and was also observed against the group of all HPV 16 related types (see Table 2).

Statistically significant cross protection was observed against cytological abnormalities associated with HPV 52 and was also observed against cytological abnormalities associated with the group of all HPV 16 related types (HPV-31, 33, 35, 52, and 58) and the group of all high risk types, excluding 16 and 18 (31, 33, 35, 39, 45, 51, 52, 56, 58, 59, 66, and 68).

TABLE A

| | | | | | % vaccine efficacy | | | |
|---|---|---|---|---|---|---|---|---|
| | Number of | | Number of | | 1 − (A/B) × | | | |
| | women | % women | women | % women | 100, adjusted for | 95% | 95% | |
| | infected | infected | infected | infected | relative size of | confidence | confidence | |
| HPV types | (vaccine | (vaccine | (placebo | (placebo | vaccine and | limits- | limits- | |
| analysed | group) | group) = A | group) | group) = B | placebo group | lower limit | upper limit | P |
| HPV 31, 35, 58 | 5 | 1.1 | 11 | 2.4 | 55.1 | −29.1 | 84.4 | 0.127 |
| HPV 31, 33, 35, | 17 | 3.8 | 24 | 5.4 | 30.3 | −29.7 | 62.6 | 0.252 |
| 52, 58 | | | | | | | | |
| HPV 45, 59 | 3 | 0.7 | 6 | 1.3 | 50.6 | −97.7 | 87.6 | 0.309 |
| HPV 31, 33, 35, | 27 | 6.3 | 40 | 9.4 | 34.6 | −6.5 | 59.9 | 0.086 |
| 39, 45, 51, 52, | | | | | | | | |
| 56, 58, 59, 66, | | | | | | | | |
| 68. | | | | | | | | |

Samples were taken at 9, 12, 15 and 18 months from patients and tested for HPV infection by the types specified above.

TABLE B
vaccine efficacy after three doses in preventing incident heterologous infections.

| | Attack rate | | | | | | | | | |
|---|---|---|---|---|---|---|---|---|---|---|
| | Vaccine | | | Placebo | | | Vaccine efficacy | | | |
| Infection Type | N | n | AR | N | n | AR | % | 95% CI | | p-value |
| HPV-16 related | 433 | 12 | 2.8 | 438 | 24 | 5.5 | 49.4 | 0.2 | 74.4 | 0.060 |
| HPV-16 related* | 423 | 29 | 6.9 | 423 | 46 | 10.9 | 37.0 | 1.6 | 59.6 | 0.052 |
| HPV-18 related | 442 | 9 | 2.0 | 449 | 16 | 3.6 | 42.9 | −27.9 | 74.5 | 0.223 |
| HPV-16/18 related | 433 | 21 | 4.9 | 438 | 41 | 9.4 | 48.2 | 13.8 | 68.9 | 0.012 |
| HPV-16/18 related* | 423 | 34 | 8.0 | 423 | 56 | 13.2 | 39.3 | 9.0 | 59.5 | 0.019 |
| High-risk** | 385 | 53 | 13.8 | 386 | 88 | 22.8 | 39.6 | 17.7 | 55.7 | 0.001 |

Vaccine efficacy against infection with HPV-16 phylogenetically related types, HPV-18 phylogenetically related types, HPV-16 and/or HPV-18 phylogenetically related types and all high-risk types exclusive of HPV-16 and HPV-18 - ATP cohort (month 6-18)
N = number of subjects in specific cohort
n = number of subjects with incident HPV infection
AR = Attack rate = n/N
95% CI = 95% confidence interval
lower limit = 1 − exp (log (arv/arp) + 1.96* sqrt (1/nv − 1/Nv + 1/np − 1/Np))
upper limit = 1 − exp (log (arv/arp) − 1.96* sqrt (1/nv − 1/Nv + 1/np − 1/Np))
when number of cases in vaccine = 0:
lower limit* = 1 − exp (log (arv*/arp*) + 1.96* sqrt (1/(nv + 0.5) − 1/(Nv + 0.5) + 1/(np + 0.5) − 1/(Np + 0.5)))
upper limit* = 1 − exp (log (arv*/arp*) − 1.96* sqrt (1/(nv + 0.5) − 1/(Nv + 0.5) + 1/(np + 0.5) − 1/(Np + 0.5)))
with:
arv = attack rate in vaccine recipients
arp = attack rate in placebo recipients
nv = number of cases in vaccine recipients
Nv = number of cases and non-cases in vaccine recipients
np = number of cases in placebo recipients
Np = number of cases and non-cases in placebo recipients
HPV-16 related: HPV-16 phylogenetically related types 35, 31, 58 without considering other HPV types
HPV-16 related*: HPV-16 phylogenetically related types 35, 31, 58, 33, 52 without considering other HPV types
HPV-18 related: HPV-18 phylogenetically related types 45, 59 without considering other HPV types
HPV-16 and/or HPV-18 related: HPV-16 and/or HPV-18 phylogenetically related types 35, 31, 58, 45, 59 without considering other HPV types
HPV-16 and/or HPV-18 related*: HPV-16 and/or HPV-18 phylogenetically related types 35, 31, 58, 33, 52, 45, 59 without considering other HPV types
**= High-risk types exclusive of HPV-16 and HPV-18

TABLE C

| | Total number | | | | | % vaccine efficacy | | | |
|---|---|---|---|---|---|---|---|---|---|
| | of number of | Number of | | Number of | | 1 − (A/B) × 100, | | | |
| | subjects with | women | % women | women | % women | adjusted for | 95% | 95% | |
| | information | infected | infected | infected | infected | relative size of | confidence | confidence | |
| HPV types | available per | (vaccine | (vaccine | (placebo | (placebo | vaccine and | limits- | limits- | |
| analysed | group | group) | group) = A | group) | group) = B | placebo group | lower limit | upper limit | P |
| HPV 31, 35, 58 | 412 | 11 | 2.7 | 26 | 6.3 | 57.9 | 15.9 | 78.9 | 0.012 |
| HPV 31, 33, 35, | 403 | 28 | 6.9 | 48 | 12.2 | 43.0 | 11.0 | 63.5 | 0.015 |
| 52, 58 | | | | | | | | | |
| HPV 45, 59 | 421 | 10 | 2.4 | 15 | 3.6 | 33.5 | −46.3 | 69.8 | 0.319 |
| HPV 31, 33, 35, | 368 | 58 | 15.8 | 90 | 25.3 | 37.7 | 16.2 | 53.6 | 0.002 |
| 39, 45, 51, 52, | | | | | | | | | |
| 56, 58, 59, 66, | | | | | | | | | |
| 68. | | | | | | | | | |

Samples were taken at 18 months from patients and tested for HPV infection by the types specified above.

TABLE 1
Efficacy against Incident Infections with 16/18 Related Types*

| | | Vaccine | | Control | | Vaccine Efficacy | |
|---|---|---|---|---|---|---|---|
| | HPV Type | N | AR | N | AR | % | P value |
| 16 related | HPV-31 | 1 | 0.2 | 10 | 2.4 | 90.0 | 0.006 |
| | HPV-33 | 6 | 1.4 | 6 | 1.4 | −0.2 | 1.000 |
| | HPV-35 | 1 | 0.2 | 3 | 0.7 | 66.5 | 0.624 |
| | HPV-52 | 6 | 1.4 | 16 | 3.9 | 63.0 | 0.031 |
| | HPV-58 | 5 | 1.2 | 5 | 1.2 | 0.0 | 1.000 |
| 18 related | HPV-45 | 0 | 0.0 | 5 | 1.2 | 100.0 | 0.031 |
| | HPV-59 | 4 | 0.9 | 2 | 0.5 | −100.5 | 0.448 |
| | All 16 related | 16 | 4.0 | 32 | 8.1 | 51.1 | 0.017 |
| | All 18 related | 4 | 1.0 | 7 | 1.7 | 43.0 | 0.384 |
| | All HR | 32 | 9.0 | 53 | 15.6 | 42.3 | 0.011 |
| | (except 16/18) | | | | | | |

*Cervical samples; ATP cohort

TABLE 2
Efficacy against Persistent Infections with 16/18 Related Types*

| | | Vaccine | | Control | | Vaccine Efficacy | |
|---|---|---|---|---|---|---|---|
| | HPV Type | N | AR | N | AR | % | P value |
| 16 related | HPV-31 | 2 | 0.48 | 9 | 2.15 | 78.5 | 0.030 |
| | HPV-33 | 3 | 0.71 | 5 | 1.18 | 40.2 | 0.476 |
| | HPV-35 | 1 | 0.24 | 1 | 0.24 | 0.4 | 0.998 |
| | HPV-52 | 5 | 1.20 | 21 | 5.10 | 77.1 | 0.001 |
| | HPV-58 | 4 | 0.95 | 6 | 1.42 | 34.1 | 0.515 |
| 18 related | HPV-45 | 1 | 0.24 | 4 | 0.94 | 75.4 | 0.174 |
| | HPV-59 | 3 | 0.71 | 0 | 0.00 | — | 0.083 |
| | All 16 related | 11 | 2.7 | 30 | 7.6 | 65.1 | 0.002 |
| | All 18 related | 4 | 1.0 | 4 | 1.0 | 1.0 | 0.989 |
| | All HR | 36 | 10.1 | 46 | 13.5 | 27.1 | 0.155 |
| | (except 16/18) | | | | | | |

*All samples; ATP cohort

TABLE 3
Efficacy against Cytological Abnormalities
ass. with 16/18 Related Types*

| | | Vaccine | | Control | | Vaccine Efficacy | |
|---|---|---|---|---|---|---|---|
| | HPV Type | N | AR | N | AR | % | P value |
| 16 related | HPV-31 | 1 | 0.24 | 5 | 1.20 | 80.1 | 0.123 |
| | HPV-33 | 2 | 0.47 | 4 | 0.94 | 49.9 | 0.686 |
| | HPV-35 | 0 | 0.00 | 2 | 0.47 | 100 | 0.499 |
| | HPV-52 | 1 | 0.24 | 11 | 2.67 | 91 | 0.003 |
| | HPV-58 | 2 | 0.47 | 2 | 0.47 | 0.2 | 1.000 |
| 18 related | HPV-45 | 0 | 0.00 | 2 | 0.47 | 100 | 0.249 |
| | HPV-59 | 4 | 0.94 | 2 | 0.47 | −101 | 0.451 |
| | All 16 related | 5 | 1.2 | 18 | 4.6 | 72.8 | 0.005 |
| | All 18 related | 4 | 1.0 | 4 | 1.0 | 0.2 | 1.000 |
| | All HR | 10 | 2.8 | 30 | 8.8 | 68.2 | <0.001 |
| | (except 16/18) | | | | | | |

*ATP cohort
In tables 1, 2 and 3,
N = number of subjects in specific cohort
AR = Attack rate = n (number of subjects with HPV either incident infection, persistent infection or cytological abnormality, as appropriate for the table)/N
% Vaccine efficacy is 1 − (A/B) × 100, adjusted for relative size of vaccine and placebo group, wherein
A = % women in vaccine group with incident infection, persistent infection or cytological abnormality, as appropriate for the table
B = % women in placebo group with incident infection, persistent infection or cytological abnormality, as appropriate for the table

### Example 2

10 groups of C57B1/6 mice were used, containing 24 mice per group.

Mice were vaccinated according to the information below in a 2 dose schedule at day 0 and day 28, using intramuscular administration (1 group of mice were both primed and boosted with NaCl, not listed below).

Vaccination was carried out using different HPV virus like particle combinations. 4 different VLPs were used: HPV 16 L1 only VLPs, HPV 18 L1 only VLPs, HPV 31 L1 only VLPs, and HPV 45 L1 only VLPs.

The VLPs were made and purified essentially according to the disclosure of WO2003077942A2, herein incorporated fully by reference.

In more detail, HPV 16 VLPs were purified using the sequential steps of: anion exchange chromatography (Di methyl amino ethyl—DMAE), anion exchange chromatography (tri methyl amino ethyl—TMAE), hydroxyapatite chromatography, filtration and another anion exchange step, this time using a Di ethyl amino ethyl (DEAE) step, followed by a final filtration. HPV 31 VLPs were made using the same sequence of steps.

HPV 18 VLPs were purified using the sequential steps of: anion exchange chromatography (Di methyl amino ethyl—DMAE), anion exchange chromatography (tri methyl amino ethyl—TMAE), hydroxyapatite chromatography, filtration and an octyl sepharose column (hydrophobic interaction chromatography), followed by a final filtration. HPV 45 VLPs were made using the same sequence of steps.

The following L1 sequences were used to make VLPs for this experiment:

| Nucleotidic Sequence of Truncated L1 from serotype 16 | |
|---|---|
| (SEQ ID NO: 3) | |
| ATGTCTCTTTGGCTGCCTAGTGAGGCCACTGTCTACTTGCCTCCTGTCCCAGTATCTAAGGTTGTAAGCA | 70 |
| CGGATGAATATGTTGCACGCACAAACATATATTATCATGCAGGAACATCCAGACTACTTGCAGTTGGACA | 140 |
| TCCCTATTTTCCTATTAAAAAACCTAACAATAACAAAATATTAGTTCCTAAAGTATCAGGATTACAATAC | 210 |
| AGGGTATTTAGAATACATTTACCTGACCCCAATAAGTTTGGTTTTCCTGACACCTCATTTTATAATCCAG | 280 |
| ATACACAGCGGCTGGTTTGGGCCTGTGTAGGTGTTGAGGTAGGTCGTGGTCAGCCATTAGGTGTGGGCAT | 350 |
| TAGTGGCCATCCTTTATTAAATAAATTGGATGACACAGAAAATGCTAGTGCTTATGCAGCAAATGCAGGT | 420 |
| GTGGATAATAGAGAATGTATATCTATGGATTACAAACAAACACAATTGTGTTTAATTGGTTGCAAACCAC | 490 |
| CTATAGGGGAACACTGGGGCAAAGGATCCCCATGTACCAATGTTGCAGTAAATCCAGGTGATTGTCCACC | 560 |
| ATTAGAGTTAATAAACACAGTTATTCAGGATGGTGATATGGTTGATACTGGCTTTGGTGCTATGGACTTT | 630 |
| ACTACATTACAGGCTAACAAAAGTGAAGTTCCACTGGATATTTGTACATCTATTTGCAAATATCCAGATT | 700 |
| ATATTAAAATGGTGTCAGAACCATATGGCGACAGCTTATTTTTTTATTTACGAAGGGAACAAATGTTTGT | 770 |
| TAGACATTTATTTAATAGGGCTGGTGCTGTTGGTGAAAATGTACCAGACGATTTATACATTAAAGGCTCT | 840 |
| GGGTCTACTGCAAATTTAGCCAGTTCAAATTATTTTCCTACACCTAGTGGTTCTATGGTTACCTCTGATG | 910 |
| CCCAAATATTCAATAAACCTTATTGGTTACAACGAGCACAGGGCCACAATAATGGCATTTGTTGGGGTAA | 980 |
| CCAACTATTTGTTACTGTTGTTGATACTACACGCAGTACAAATATGTCATTATGTGCTGCCATATCTACT | 1050 |
| TCAGAAACTACATATAAAAATACTAACTTTAAGGAGTACCTACGACATGGGGAGGAATATGATTTACAGT | 1120 |
| TTATTTTTCAACTGTGCAAAATAACCTTAACTGCAGACGTTATGACATACATACATTCTATGAATTCCAC | 1190 |
| TATTTTCGAGGACTGGAATTTTGGTCTACAACCTCCCCCAGGAGGCACACTAGAAGATACTTATAGGTTT | 1260 |
| GTAACATCCCAGGCAATTGCTTGTCAAAAACATACACCTCCAGCACCTAAAGAAGATCCCCTTAAAAAAT | 1330 |
| ACACTTTTTGGGAAGTAAATTTAAAGGAAAAGTTTTCTGCAGACCTAGATCAGTTTCCTTTAGGACGCAA | 1400 |
| ATTTTTACTACAATAA | 1416 |
| Nucleotidic Sequence of Truncated L1 from serotype 18 | |
| (SEQ ID NO: 4) | |
| ATGGCTTTGTGGCGGCCTAGTGACAATACCGTATATCTTCCACCTCCTTCTGTGGCAAGAGTTGTAAATA | 70 |
| CCGATGATTATGTGACTCGCACAAGCATATTTTATCATGCTGGCAGCTCTAGATTATTAACTGTTGGTAA | 140 |
| TCCATATTTTAGGGTTCCTGCAGGTGGTGGCAATAAGCAGGATATTCCTAAGGTTTCTGCATACCAATAT | 210 |
| AGAGTATTTAGGGTGCAGTTACCTGACCCAAATAAATTTGGTTTACCTGATAATAGTATTTATAATCCTG | 280 |
| AAACACAACGTTTAGTGTGGGCCTGTGTTGGAGTGGAAATTGGCCGTGGTCAGCCTTTAGGTGTTGGCCT | 350 |
| TAGTGGGCATCCATTTTATAATAAATTAGATGACACTGAAAGTTCCCATGCCGCCACGTCTAATGTTTCT | 420 |
| GAGGACGTTAGGGACAATGTGTCTGTAGATTATAAGCAGACACAGTTATGTATTTTGGGCTGTGCCCCTG | 490 |
| CTATTGGGGAACACTGGGCTAAAGGCACTGCTTGTAAATCGCGTCCTTTATCACAGGGCGATTGCCCCCC | 560 |
| TTTAGAACTTAAAAACACAGTTTTGGAAGATGGTGATATGGTAGATACTGGATATGGTGCCATGGACTTT | 630 |
| AGTACATTGCAAGATACTAAATGTGAGGTACCATTGGATATTTGTCAGTCTATTTGTAAATATCCTGATT | 700 |
| ATTTACAAATGTCTGCAGATCCTTATGGGGATTCCATGTTTTTTTGCTTACGGCGTGAGCAGCTTTTTGC | 770 |
| TAGGCATTTTTGGAATAGGGCAGGTACTATGGGTGACACTGTGCCTCCATCCTTATATATTAAAGGCACA | 840 |
| GGTATGCGTGCTTCACCTGGCAGCTGTGTGTATTCTCCCTCTCCAAGTGGCTCTATTGTTACCTCTGACT | 910 |
| CCCAGTTGTTTAATAAACCATATTGGTTACATAAGGCACAGGGTCATAACAATGGTGTTTGCTGGCATAA | 980 |
| TCAATTATTTGTTACTGTGGTAGATACCACTCGCAGTACCAATTTAACAATATGTGCTTCTACACAGTCT | 1050 |
| CCTGTACCTGGGCAATATGATGCTACCAAATTTAAGCAGTATAGCAGACATGTTGAGGAATATGATTTGC | 1120 |
| AGTTTATTTTTCAGTTGTGTACTATTACTTTAACTGCAGATGTTATGTCCTATATTCATAGTATGAATAG | 1190 |
| CAGTATTTTAGAGGATTGGAACTTTGGTGTTCCCCCCCCGCCAACTACTAGTTTGGTGGATACATATCGT | 1260 |
| TTTGTACAATCTGTTGCTATTACCTGTCAAAAGGATGCTGCACCGGCTGAAAATAAGGATCCCTATGATA | 1330 |
| AGTTAAAGTTTTGGAATGTGGATTTAAAGGAAAAGTTTTCTTTAGACTTAGATCAATATCCCCTTGGACG | 1400 |
| TAAATTTTTGGTTCAGTAA | 1419 |
| Nucleotidic Sequence of Truncated L1 from serotype 31 | |
| (SEQ ID NO: 5) | |
| ATGTCTCTGTGGCGGCCTAGCGAGGCTACTGTCTACTTACCACCTGTCCCAGTGTCTAAAGTTGTAAGCA | 70 |
| CGGATGAATATGTAACACGAACCAACATATATTATCACGCAGGCAGTGCTAGGCTGCTTACAGTAGGCCA | 140 |
| TCCATATTATTCCATACCTAAATCTGACAATCCTAAAAAAATAGTTGTACCAAAGGTGTCAGGATTACAA | 210 |
| TATAGGGTATTTAGGGTTCGTTTACCAGATCCAAACAAATTTGGATTTCCTGATACATCTTTTTATAATC | 280 |
| CTGAAACTCAACGCTTAGTTTGGGCCTGTGTTGGTTTAGAGGTAGGTCGCGGGCAGCCATTAGGTGTAGG | 350 |
| TATTAGTGGTCATCCATTATTAAATAAATTTGATGACACTGAAAACTCTAATAGATATGCCGGTGGTCCT | 420 |
| GGCACTGATAATAGGCAATGTATATCAATGGATTATAAACAAACACAACTGTGTTTACTTGGTTGCAAAC | 490 |
| CACCTATTGGAGAGCATTGGGGTAAAGGTAGTCCTTGTAGTAACAATGCTATTACCCCTGGTGATTGTCC | 560 |
| TCCATTAGAATTAAAAAATTCAGTTATACAAGATGGGGATATGGTTGATACAGGCTTTGGAGCTATGGAT | 630 |
| TTTACTGCTTTACAAGACACTAAAAGTAATGTTCCTTTGGACATTTGTAATTCTATTTGTAAATATCCAG | 700 |
| ATTATCTTAAAATGGTTGCTGAGCCATATGGCGATACATTATTTTTTTATTTACGTAGGGAACAAATGTT | 770 |
| TGTAAGGCATTTTTTTAATAGATCAGGCACGGTTGGTGAATCGGTCCCTACTGACTTATATATTAAAGGC | 840 |
| TCCGGTTCAACAGCTACTTTAGCTAACAGTACATACTTTCCTACACCTAGCGGCTCCATGGTTACTTCAG | 910 |
| ATGCACAAATTTTTAATAAACCATATTGGATGCAACGTGCTCAGGGACACAATAATGGTATTTGTTGGGG | 980 |
| CAATCAGTTATTTGTTACTGTGGTAGATACCACACGTAGTACCAATATGTCTGTTTGTGCTGCAATTGCA | 1050 |
| AACAGTGATACTACATTTAAAAGTAGTAATTTTAAAGAGTATTTAAGACATGGTGAGGAATTTGATTTAC | 1120 |
| AATTTATATTTCAGTTATGCAAAATAACATTATCTGCAGACATAATGACATATATTCACAGTATGAATCC | 1190 |
| TGCTATTTTGGAAGATTGGAATTTTGGATTGACCACACCTCCCTCAGGTTCTTTGGAGGATACCTATAGG | 1260 |
| TTTGTCACCTCACAGGCCATTACATGTCAAAAAACTGCCCCCCAAAAGCCCAAGGAAGATCCATTTAAAG | 1330 |
| ATTATGTATTTTGGGAGGTTAATTTAAAAGAAAAGTTTTCTGCAGATTTAGATCAGTTTCCACTGGGTCG | 1400 |
| CAAATTTTTATTACAGTAA | 1419 |
| Nucleotidic Sequence of Truncated L1 from serotype 45 | |
| (SEQ ID NO: 6) | |
| ATGGCTTTGTGGCGGCCTAGTGACAGTACGGTATATCTTCCACCACCTTCTGTGGCCAGAGTTGTCAACA | 70 |
| CTGATGATTATGTGTCTCGCACAAGCATATTTTACCATGCAGGCAGTTCCCGATTATTAACTGTAGGCAA | 140 |
| TCCATATTTTAGGGTTGTACCTAGTGGTGCAGGTAATAAACAGGCTGTTCCTAAGGTATCCGCATATCAG | 210 |
| TATAGGGTGTTTAGAGTAGCTTTACCCGATCCTAATAAATTTGGATTACCTGATTCTACTATATATAATC | 280 |
| CTGAAACACAACGTTTGGTTTGGGCATGTGTAGGTATGGAAATTGGTCGTGGGCAGCCTTTAGGTATTGG | 350 |
| CCTAAGTGGCCATCCATTTTATAATAAATTGGATGATACAGAAAGTGCTCATGCGGCTACGGCTGTTGTT | 420 |
| ACGCAGGATGTTAGGGATAATGTGTCAGTTGATTATAAGCAAACACAGCTGTGTATTTTAGGTTGTGTAC | 490 |
| CTGCTATTGGTGAGCACTGGGCCAAGGGCACACTTTGTAAACCTGCACAATTGCAGCCTGGTGACTGTCC | 560 |
| TCCTTTGGAACTTAAAAACACCATTATTGAGGATGGTGATATGGTGGATACAGGTTATGGGGCAATGGAT | 630 |
| TTTAGTACATTGCAGGATACAAAGTGCGAGGTTCCATTAGACATTTGTCAATCCATCTGTAAATATCCAG | 700 |
| ATTATTTGCAAATGTCTGCTGATCCCTATGGGGATTCTATGTTTTTTTGCCTACGCCGTGAACAACTGTT | 770 |
| TGCAAGACATTTTTGGAATAGGGCAGGTGTTATGGGTGACACAGTACCTACAGACCTATATATTAAAGGC | 840 |
| ACTAGCGCTAATATGCGTGAAACCCCTGGCAGTTGTGTGTATTCCCCTTCTCCCAGTGGCTCTATTATTA | 910 |
| CTTCTGATTCTCAATTATTTAATAAGCCATATTGGTTACATAAGGCCCAGGGCCATAACAATGGTATTTG | 980 |
| TTGGCATAATCAGTTGTTTGTTACTGTAGTGGACACTACCCGCAGTACTAATTTAACATTATGTGCCTCT | 1050 |
| ACACAAAATCCTGTGCCAGGTACATATGATCCTACTAAGTTTAAGCACTATAGTAGACATGTGGAGGAAT | 1120 |
| ATGATTTACAGTTTATTTTTCAGTTGTGCACTATTACTTTAACTGCAGAGGTTATGTCATATATCCATAG | 1190 |
| TATGAATAGTAGTATATTGGAAAATTGGAATTTTGGTGTACCTCCACCACCTACTACAAGTTTAGTGGAT | 1260 |
| ACATATCGTTTTGTGCAATCAGTTGCTGTTACCTGTCAAAAGGATACTACACCTCCAGAAAAGCAGGATC | 1330 |
| CATATGATAAATTAAAGTTTTGGACTGTTGACCTAAAGGAAAAATTTTCCTCCGATTTGGATCAATATCC | 1400 |
| CCTTGGTCGAAAGTTTTTAGTTCAGTAA | 1428 |

Each antigen was used at a dose of 2 μg. HPV 16, 18 and 45 VLPS were adsorbed onto

The adjuvant used was termed AS04D, at 1/10 of the human dose, which is 5 μg 3D MPL and 50 μg aluminium hydroxide in total for each vaccine.

HPV virus like particles were combined with aluminium hydroxide and 3D MPL as disclosed in WO00/23105.

For example, in the tetravalent 16, 18, 31, 45 vaccine, 2 μg of each of HPV 16 VLPs was adsorbed onto 5 μg aluminium hydroxide. For HPV 18 and HPV 45 the same was done. For HPV 31 VLPS the 2 μg VLP was adsorbed onto 2.5 μg aluminium hydroxide.

Separately 5 μg 3D-MPL was adsorbed onto 17.5 μg aluminium hydroxide.

The 3D-MPL and VLPs were then mixed, and additional aluminium hydroxide added to result in 50 μg per vaccine dose.

### Vaccination Schedule:

| First composition | Second composition |
|---|---|
| VLPs 16/18 AS04D | VLPs 16/18 AS04D |
| VLPs 16/18/31/45 AS04D | VLPs 16/18/31/45 AS04D |
| NaCl | VLPs 31/45 AS04D |

### Read Outs

Antibody titres were determined using classical ELISA techniques well known in the art at day 14 post the initial vaccination and second vaccination (post I and post II respectively).

Intracellular staining (ICS, Roederer et al. 2004 Clin. Immunol. 110: 199) was carried out at 14 days post II to assess CMI responses.

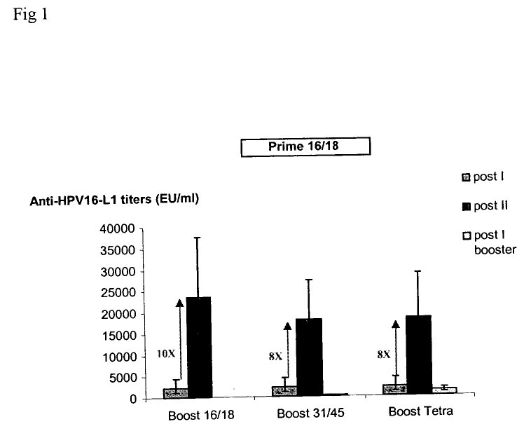

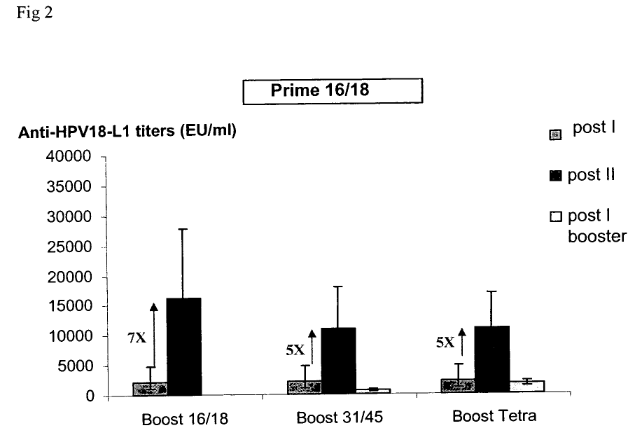

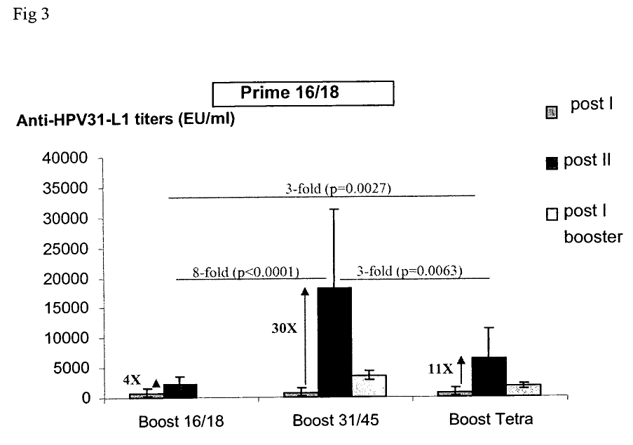

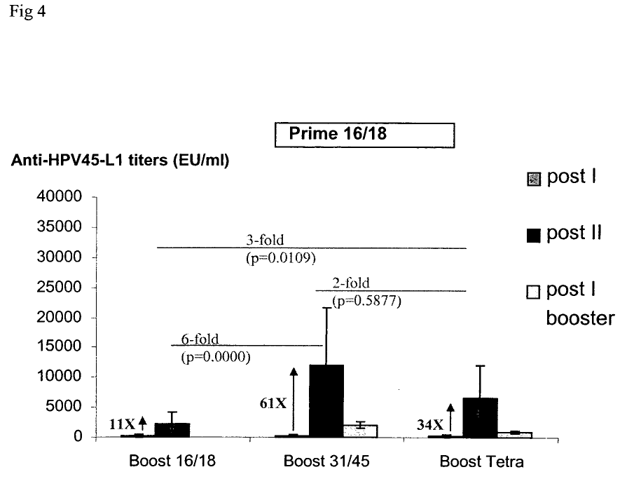

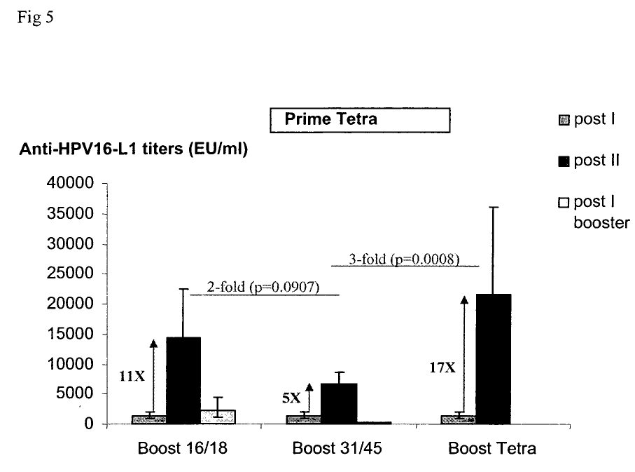

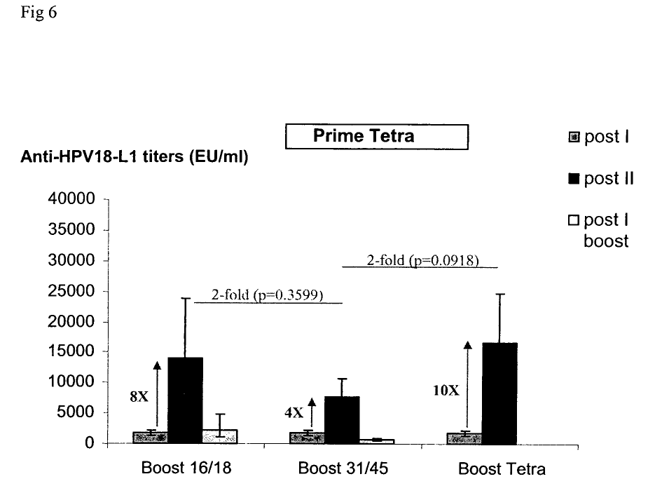

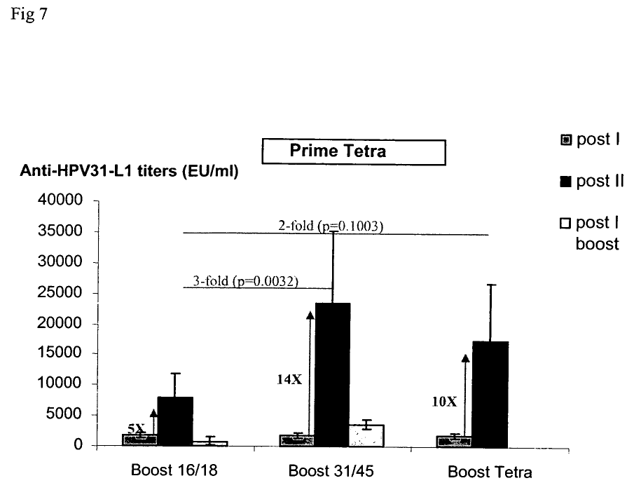

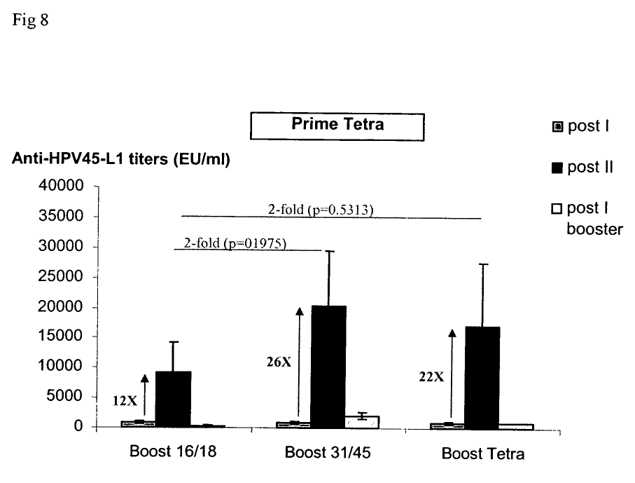

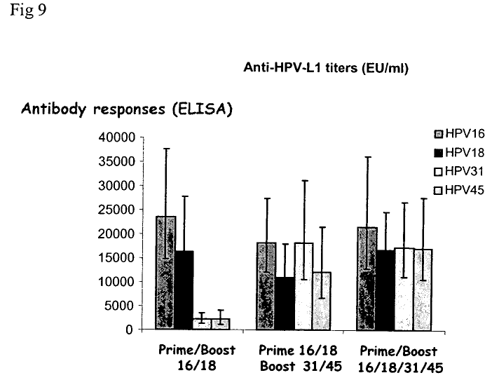

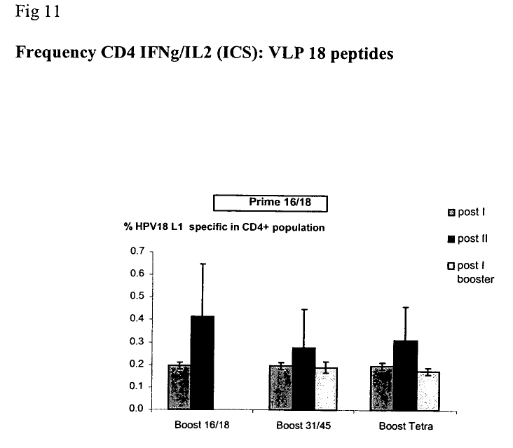

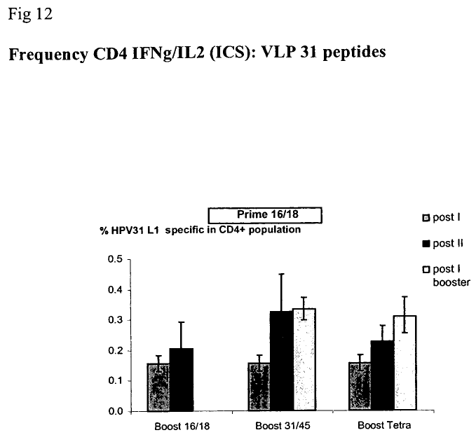

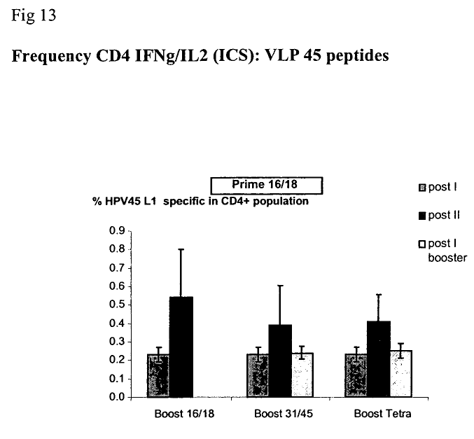

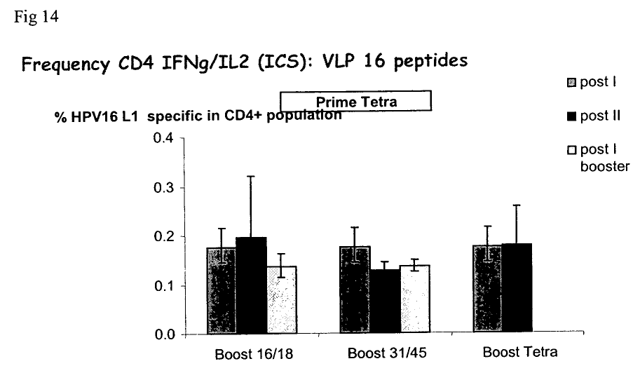

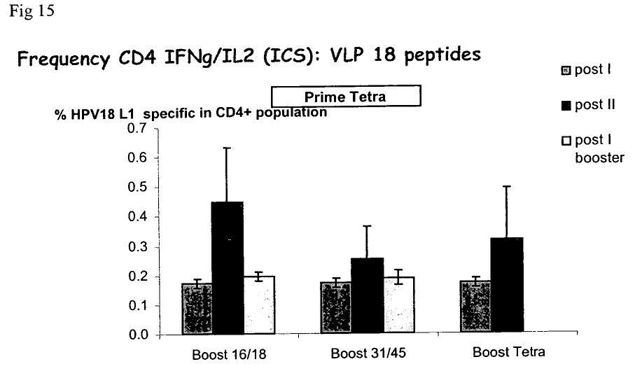

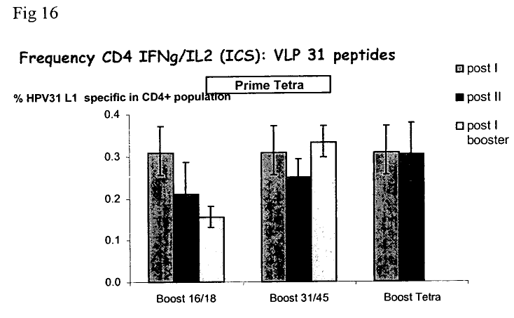

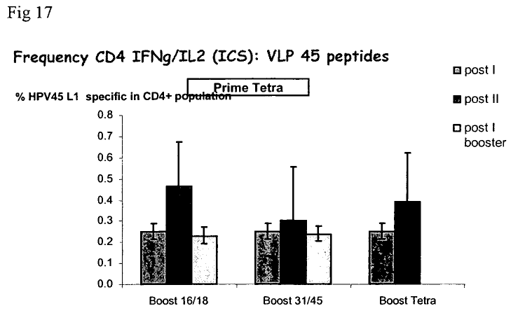

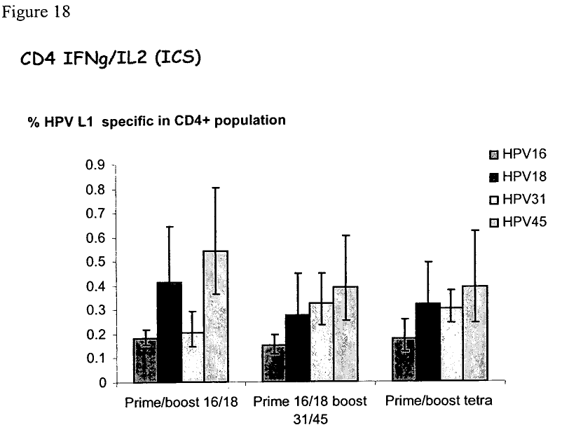

Results are given in FIGS. 1-18 below. In these figures the post I and post II data are measurements after vaccine dose I and II respectively. The bar given as “post I booster” reflects the group in which NaCl was given first, followed by either a bivalent or tetravalent vaccine dose at day 28.

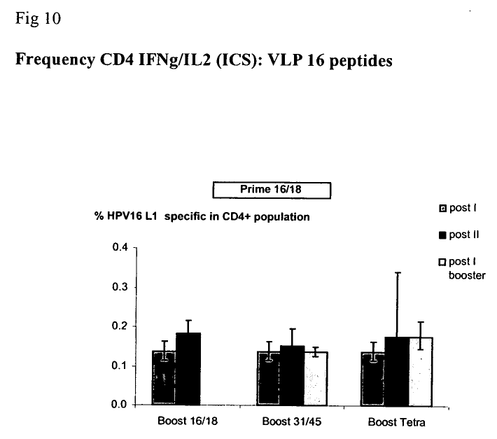

FIGS. 1-9 illustrate the antibody responses generated by different vaccination regimes. FIGS. 10-18 illustrate the CMI responses generated by different vaccination regimes.

### CONCLUSIONS

1—A heterologous booster comprised of HPV 31 and 45 L1 VLPs is effective in boosting homologous HPV 16 and 18 L1 VLP responses induced by HPV 16 and 18 L1 VLP priming.

2—A heterologous booster comprised of HPV 31 and 45 L1 VLPs is effective in boosting heterologous HPV 31 and 45 L1 VLP responses induced by HPV 16 and 18 L1 VLP priming.

1-10. (canceled)

11. A vaccination schedule for protection against HPV infection and/or disease, the schedule comprising delivering a first vaccine comprising an L1 protein or immunogenic fragment thereof from HPV 16 and an L1 protein or immunogenic fragment thereof from HPV 18, and delivering a second vaccine comprising an L1 protein or immunogenic fragment thereof from at least one other oncogenic HPV type provided that the at least one other oncoqenic HPV type is not HPV 16 or HPV 18, wherein the first and second vaccines may be delivered in any order and.

12. A method for prevention of HPV infection and/or disease, the method comprising the steps of delivering a first vaccine comprising an L1 protein or immunogenic fragment thereof from HPV 16 and an L1 protein or immunogenic fragment thereof from HPV 18, and delivering a second vaccine comprising an L1 protein or immunogenic fragment thereof from at least one other oncogenic HPV type provided that the at least one other oncogenic HPV type is not HPV 16 or HPV 18, wherein the first and second vaccines may be delivered in any order.

13. The method according to claim 12, wherein the first vaccine further comprises an L1 protein or immunogenic fragment thereof from HPV 33.

14. The method according to claim 13 wherein the first vaccine additionally comprises L1 protein or immunogenic fragment thereof from HPV 58.

15. The method according to claim 12 wherein the second vaccine comprises an L1 protein or immunogenic fragment thereof selected from at least one member of the group consisting of HPV 31, HPV 45 and HPV 52.

16. The method according to claim 15, wherein the second vaccine comprises an L1 protein or immunogenic fragment thereof from HPV 31 and an L1 protein or immunogenic fragment thereof from HPV 45.

17. The method according to claim 15, wherein the second vaccine comprises an L1 protein or immunogenic fragment thereof from HPV 31, an L1 protein or immunogenic fragment thereof from HPV 45 and an L1 protein or immunogenic fragment thereof from HPV 52.

18-19. (canceled)

20. A vaccine composition comprising an HPV 31 L1 protein or immunogenic fragment thereof and an HPV 45 L1 protein or immunogenic fragment thereof, wherein the vaccine does not contain an HPV 16 protein or immunogenic fragment thereof or an HPV 18 L1 protein or immunogenic fragments thereof.

21. The vaccine composition according to claim 20 further comprising an HPV 52 L1 protein or immunogenic fragment thereof.

22. A kit comprising a first vaccine comprising an L1 protein or immunogenic fragment thereof from HPV 16 and an L1 protein or immunogenic fragment thereof from HPV 18, and a second vaccine comprising an L1 protein or immunogenic fragment thereof from at least one other oncogenic HPV type provided that the at least one other oncogenic HPV type is not HPV 16 or HPV 18.

23. The kit according to claim 22 wherein the second vaccine component comprises an L1 protein or immunogenic fragment thereof from HPV 31 and an L1 protein or immunogenic fragment thereof from HPV 45.

24. The method according to claim 12 wherein at least one of the HPV L1 proteins or immunogenic fragments thereof is in the form of a virus like particle.

25. The method according to claim 12 wherein the first vaccine or the second vaccine or the first and second vaccines comprises an adjuvant.

26. The method according to claim 25 wherein the adjuvant comprises 3D-MPL.

27. The method according to claim 25 wherein the adjuvant comprises an aluminum salt.

28. The method according to claim 25 wherein the adjuvant comprises an aluminum salt and 3D MPL.

29. The method according to claim 26 wherein the adjuvant further comprises an oil-in-water emulsion adjuvant.

30. The method according to claim 29 wherein the oil-in-water emulsion comprises a metabolisable oil, a sterol and an emulsifying agent.

31. The method according to claim 12 wherein the first vaccine additionally comprises an L1 protein or immunogenic fragment thereof from HPV 33 and an L1 protein or immunogenic fragment thereof from HPV 58.

32. The method according to claim 12 wherein delivery of the first and second vaccines is separated by a suitable time interval.

33. The vaccine composition according to claim 20 further comprising an adjuvant.

34. The vaccine composition according to claim 33 wherein the adjuvant comprises 3D-MPL.

35. The vaccine composition according to claim 33 wherein the adjuvant comprises an aluminum salt.

36. The vaccine composition according to claim 33 wherein the adjuvant comprises an aluminum salt and 3D MPL.

37. The vaccine composition according to claim 34 wherein the adjuvant further comprises an oil-in-water emulsion adjuvant.

38. The vaccine composition according to claim 37 wherein the oil-in-water emulsion comprises a metabolisable oil, a sterol and an emulsifying agent.


SEQUENCE LISTING (6 sequences)

SEQ ID NO: 1 - protein, 471 aa, HUMAN PAPILLOMA VIRUS L1 PARTIAL SEQUENCE hpv 16
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQ

SEQ ID NO: 2 - protein, 472 aa, HUMAN PAPILLOMA VIRUS L1 PARTIAL SEQUENCE hpv 18
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQ

SEQ ID NO: 3 - DNA, 1416 bp, HUMAN PAPILLLOMA VIRUS PARTIAL L1 SEQUENCE hpv 16
atgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaataa

SEQ ID NO: 4 - DNA, 1419 bp, HUMAN PAPILLLOMA VIRUS PARTIAL L1 SEQUENCE hpv 18
atggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgataatagtatttataatcctgaaacacaacgtttagtgtgggcctgtgttggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagggcaggtactatgggtgacactgtgcctccatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcagtaa

SEQ ID NO: 5 - DNA, 1419 bp, HUMAN PAPILLLOMA VIRUS PARTIAL L1 SEQUENCE hpv 31
atgtctctgtggcggcctagcgaggctactgtctacttaccacctgtcccagtgtctaaagttgtaagcacggatgaatatgtaacacgaaccaacatatattatcacgcaggcagtgctaggctgcttacagtaggccatccatattattccatacctaaatctgacaatcctaaaaaaatagttgtaccaaaggtgtcaggattacaatatagggtatttagggttcgtttaccagatccaaacaaatttggatttcctgatacatctttttataatcctgaaactcaacgcttagtttgggcctgtgttggtttagaggtaggtcgcgggcagccattaggtgtaggtattagtggtcatccattattaaataaatttgatgacactgaaaactctaatagatatgccggtggtcctggcactgataatagggaatgtatatcaatggattataaacaaacacaactgtgtttacttggttgcaaaccacctattggagagcattggggtaaaggtagtccttgtagtaacaatgctattacccctggtgattgtcctccattagaattaaaaaattcagttatacaagatggggatatggttgatacaggctttggagctatggattttactgctttacaagacactaaaagtaatgttcctttggacatttgtaattctatttgtaaatatccagattatcttaaaatggttgctgagccatatggcgatacattatttttttatttacgtagggaacaaatgtttgtaaggcatttttttaatagatcaggcacggttggtgaatcggtccctactgacttatatattaaaggctccggttcaacagctactttagctaacagtacatactttcctacacctagcggctccatggttacttcagatgcacaaatttttaataaaccatattggatgcaacgtgctcagggacacaataatggtatttgttggggcaatcagttatttgttactgtggtagataccacacgtagtaccaatatgtctgtttgtgctgcaattgcaaacagtgatactacatttaaaagtagtaattttaaagagtatttaagacatggtgaggaatttgatttacaatttatatttcagttatgcaaaataacattatctgcagacataatgacatatattcacagtatgaatcctgctattttggaagattggaattttggattgaccacacctccctcaggttctttggaggatacctataggtttgtcacctcacaggccattacatgtcaaaaaactgccccccaaaagcccaaggaagatccatttaaagattatgtattttgggaggttaatttaaaagaaaagttttctgcagatttagatcagtttccactgggtcgcaaatttttattacagtaa

SEQ ID NO: 6 - DNA, 1428 bp, HUMAN PAPILLLOMA VIRUS PARTIAL L1 SEQUENCE HPV45
atggctttgtggcggcctagtgacagtacggtatatcttccaccaccttctgtggccagagttgtcaacactgatgattatgtgtctcgcacaagcatattttaccatgcaggcagttcccgattattaactgtaggcaatccatattttagggttgtacctagtggtgcaggtaataaacaggctgttcctaaggtatccgcatatcagtatagggtgtttagagtagctttacccgatcctaataaatttggattacctgattctactatatataatcctgaaacacaacgtttggtttgggcatgtgtaggtatggaaattggtcgtgggcagcctttaggtattggcctaagtggccatccattttataataaattggatgatacagaaagtgctcatgcggctacggctgttgttacgcaggatgttagggataatgtgtcagttgattataagcaaacacagctgtgtattttaggttgtgtacctgctattggtgagcactgggccaagggcacactttgtaaacctgcacaattgcagcctggtgactgtcctcctttggaacttaaaaacaccattattgaggatggtgatatggtggatacaggttatggggcaatggattttagtacattgcaggatacaaagtgcgaggttccattagacatttgtcaatccatctgtaaatatccagattatttgcaaatgtctgctgatccctatggggattctatgtttttttgcctacgccgtgaacaactgtttgcaagacatttttggaatagggcaggtgttatgggtgacacagtacctacagacctatatattaaaggcactagcgctaatatgcgtgaaacccctggcagttgtgtgtattccccttctcccagtggctctattattacttctgattctcaattatttaataagccatattggttacataaggcccagggccataacaatggtatttgttggcataatcagttgtttgttactgtagtggacactacccgcagtactaatttaacattatgtgcctctacacaaaatcctgtgccaggtacatatgatcctactaagtttaagcactatagtagacatgtggaggaatatgatttacagtttatttttcagttgtgcactattactttaactgcagaggttatgtcatatatccatagtatgaatagtagtatattggaaaattggaattttggtgtacctccaccacctactacaagtttagtggatacatatcgttttgtgcaatcagttgctgttacctgtcaaaaggatactacacctccagaaaagcaggatccatatgataaattaaagttttggactgttgacctaaaggaaaaattttcctccgatttggatcaatatccccttggtcgaaagtttttagttcagtaa